(19) United States
(12) Patent Application Publication
(10) Pub. No.: US 2007/0243176 A1
(43) Pub. Date: Oct. 18, 2007

(54) Human genes and gene expression products

(76) Inventors: Jaime Escobedo, Alamo, CA (US); Pablo Dominguez Garcia, San Francisco, CA (US); Julie Sudduth-Klinger, Kensington, CA (US); Christoph Reinhard, Alameda, CA (US); Filippo Randazzo, Oakland, CA (US); George Lamson, Moraga, CA (US); Elizabeth M. Scott, Sonoma, CA (US); Guozhong Zhang, Fremont, CA (US); Altaf Kassam, Oakland, CA (US); David Pot, Arlington, VA (US); Radoje Drmanac, Palo Alto, CA (US); Ivan Labat, Mountain View, CA (US); Birgit Stache-Crain, Sunnyvale, CA (US)

Correspondence Address: Chiron Corporation - Intellectual;Property - R440, P.O. Box 8097, Emeryville, CA, 94662, US

(21) Appl. No.: 10/615,618

(22) Filed: Jul. 7, 2003

Related U.S. Application Data
(63) Continuation of application No. 09/932,076, filed on Aug. 16, 2001, now abandoned.
(60) Provisional application No. 60/226,326, filed on Aug. 16, 2000.

(51) Int. Cl. A61K 48/00; C07H 21/02; C07H 21/04; C07K 14/47; C07K 16/18; C12N 15/85; C12N 5/10; C12P 21/00; C12Q 1/68

(52) U.S. Cl. 424/93.21; 435/320.1; 435/325; 435/6; 435/69.1; 530/350; 530/387.9; 536/23.1

(57) ABSTRACT

This invention relates to novel human polynucleotides and variants thereof, their encoded polypeptides and variants thereof, to genes corresponding to these polynucleotides and to proteins expressed by the genes. The invention also relates to diagnostic and therapeutic agents employing such novel human polynucleotides, their corresponding genes or gene products, e.g., these genes and proteins, including probes, antisense constructs, and antibodies.

### CROSS-REFERENCE TO RELATED APPLICATION

The application claims the benefit of earlier filed U.S. provisional application Ser. No. 60/226,326, filed Aug. 16, 2000, which application is incorporated herein by reference in its entirety.

### FIELD OF THE INVENTION

The present invention relates to polynucleotides of human origin, particularly in human colon, breast, prostate, and/or lung tissue, and the encoded gene products.

### BACKGROUND OF THE INVENTION

Identification of novel polynucleotides, particularly those that encode an expressed gene product, is important in the advancement of drug discovery, diagnostic technologies, and the understanding of the progression and nature of complex diseases such as cancer. Identification of genes expressed in different cell types isolated from sources that differ in disease state or stage, developmental stage, exposure to various environmental factors, the tissue of origin, the species from which the tissue was isolated, and the like is key to identifying the genetic factors that are responsible for the phenotypes associated with these various differences.

This invention provides novel human polynucleotides, the polypeptides encoded by these polynucleotides, and the genes and proteins corresponding to these novel polynucleotides.

### SUMMARY OF THE INVENTION

This invention relates to novel human polynucleotides and variants thereof, their encoded polypeptides and variants thereof, to genes corresponding to these polynucleotides and to proteins expressed by the genes. The invention also relates to diagnostics and therapeutics comprising such novel human polynucleotides, their corresponding genes or gene products, including probes, antisense nucleotides, and antibodies. The polynucleotides of the invention correspond to a polynucleotide comprising the sequence information of at least one of SEQ ID NOS: 1-6010.

Various aspects and embodiments of the invention will be readily apparent to the ordinarily skilled artisan upon reading the description provided herein.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

It must be noted that as used herein and in the appended claims, the singular forms “a,” “and,” and “the” include plural referents unless the context clearly dictates otherwise. Thus, for example, reference to “a polynucleotide” includes a plurality of such polynucleotides and reference to “the colon cancer cell” includes reference to one or more cells and equivalents thereof known to those skilled in the art, and so forth.

The publications and applications discussed herein are provided solely for their disclosure prior to the filling date of the present application. Nothing herein is to be construed as an admission that the present invention is not entitled to antedate such publication by virtue of prior invention. Further, the dates of publication provided may be different from the actual publication dates which may need to be independently confirmed.

Definitions

The terms “polynucleotide” and “nucleic acid,” used interchangeably herein, refer to a polymeric forms of nucleotides of any length, either ribonucleotides or deoxynucleotides. Thus, these terms include, but are not limited to, single-, double-, or multi-stranded DNA or RNA, genomic DNA, cDNA, DNA-RNA hybrids, branched nucleic acid (see, e.g., U.S. Pat. Nos. 5,124,246; 5,710,264; and 5,849,481), or a polymer comprising purine and pyrimidine bases or other natural, chemically or biochemically modified, non-natural, or derivatized nucleotide bases. These terms further include, but are not limited to, mRNA or cDNA that comprise intronic sequences (see, e.g., Niwa et al. (1999) Cell 99(7):691-702). The backbone of the polynuclotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. Peyrottes et al. (1996) Nucl. Acids Res. 24:1941-1848; Chaturvedi et al. (1996) Nucl. Acids Res. 24:2318-2323. A polynucleotide may comprise modified nucleotides, such as methylated nucleotides and nucleotide analogs, uracyl, other sugars, and linking groups such as fluororibose and thioate, and nucleotide branches. The sequence of nucleotides may be, interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or n solid support.

The terms “polypeptide” and “protein,” used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins, and the like.

“Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

“Sample” or “biological sample” as used herein encompasses a variety of sample types, and are generally meant to refer to samples of biological fluids or tissues, particularly samples obtained from tissues, especially from cells of the type associated with a disease or condition for which a diagnostic application is designed (e.g., ductal adenocarcinoma), and the like. “Sample” or “biological sample” are meant to encompass blood and other liquid samples of biological origin, solid tissue samples, such as a biopsy specimen or tissue cultures or cells derived therefrom and the progeny thereof. These terms encompass samples that have been manipulated in any way after their procurement as well as derivatives and fractions of samples, where the samples may be maniuplated by, for example, treatment with reagents, solubilization, or enrichment for certain components. The terms also encompass clinical samples, and also includes cells in cell culture, cell supernatants, cell lysates, serum, plasma, biological fluids, and tissue samples. Where the sample is solid tissue; the cells of the tissue can be dissociated or tissue sections can be analyzed.

The terms “treatment,” “treating,” “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans. Other subjects may include cattle, dogs, cats, guinea pigs, rabbits, rats, mice, horses, and so on.

As used herein the term “isolated” refers to a polynucleotide, a polypeptide, an antibody, or a host cell that is in an environment different from that in which the polynucleotide, the polypeptide, the antibody, or the host cell naturally occurs. A polynucleotide, a polypeptide, an antibody, or a host cell which is isolated is generally substantially purified. As used herein, the term “substantially purified” refers to a compound (e.g., either a polynucleotide or a polypeptide or an antibody) that is removed from its natural environment and is at least 60% free, preferably 75% free, and most preferably 90% free from other components with which it is naturally associated. Thus, for example, a composition containing A is “substantially free of” B when at least 85% by weight of the total A+B in the composition is A. Preferably, A comprises at least about 90% by weight of the total of A+B in the composition, more preferably at least about 95% or even 99% by weight.

A “host cell,” as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The terms “cancer,” “neoplasm,” “tumor,” and “carcinoma,” are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, metastatic, and non-metastatic cells. Detection of cancerous cell is of particular interest.

The use of “e”, as in 10e-3, indicates that the number to the left of “e” is raised to the power of the number to the right of “e” (thus, 10e-3 is 10⁻³).

The term “heterologous” as used herein in the context of, for example, heterologous nucleic acid or amino acid sequences, heterologous polypeptides, or heterologous nucleic acid, is meant to refer to material that originates from a source different from that with which it is joined or associated. For example, two-DNA sequences are heterologous to one another if the sequences are from different genes or from different species. A recombinant host cell containing a sequence that is heterologous to the host cell can be, for example, a bacterial cell containing a sequence encoding a human polypeptide.

The invention relates to polynucleotides comprising the disclosed nucleotide sequences, to full length cDNA, mRNA, genomic sequences, and genes corresponding to these sequences and degenerate variants thereof, and to polypeptides encoded by the polynucleotides of the invention and polypeptide variants. The following detailed description describes the polynucleotide compositions encompassed by the invention, methods for obtaining cDNA or genomic DNA encoding a full-length gene product, expression of these polynucleotides and genes, identification of structural motifs of the polynucleotides and genes, identification of the function of a gene product encoded by a gene corresponding to a polynucleotide of the invention, use of the provided polynucleotides as probes and in mapping and in tissue profiling, use of the corresponding polypeptides and other gene products to raise antibodies, and use of the polynucleotides and their encoded gene products for therapeutic and diagnostic purposes.

Polynucleotide Compositions

The scope of the invention with respect to polynucleotide compositions includes, but is not necessarily limited to, polynucleotides having a sequence set forth in any one of SEQ ID NOS: 1-6010; polynucleotides obtained from the biological materials described herein or other biological sources (particularly human sources) by hybridization under stringent conditions (particularly conditions of high stringency); genes corresponding to the provided polynucleotides; variants of the provided polynucleotides and their corresponding genes, particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other nucleic acid compositions contemplated by and within the scope of the present invention will be readily apparent to one of ordinary skill in the art when provided with the disclosure here. “Polynucleotide” and “nucleic acid” as used herein with reference to nucleic acids of the composition is not intended to be limiting as to the length or structure of the nucleic acid unless specifically indicated.

The invention features polynucleotides that are expressed in human tissue, especially human colon, prostate, breast, lung and/or endothelial tissue. Novel nucleic acid compositions of the invention of particular interest comprise a sequence set forth in any one of SEQ ID NOS: 1-6010 or an identifying sequence thereof. An “identifing sequence” is a contiguous sequence of residues at least about 10 nt to about 20 nt in length, usually at least about 50 nt to about 100 nt in length, that uniquely identifies a polynucleotide sequence, e.g., exhibits less than 90%, usually less than about 80% to about 85% sequence identity to any contiguous nucleotide sequence of more than about 20 nt. Thus, the subject novel nucleic acid compositions include fall length cDNAs or mRNAs that encompass an identifying sequence of contiguous nucleotides from any one of SEQ ID NOS: 1-6010.

The polynucleotides of the invention also include polynucleotides having sequence similarity or sequence identity. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under stringent conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences (SEQ ID NOS: 1-6010) under stringent hybridization conditions. By using probes, particularly labeled probes of DNA sequences, one can isolate homologous or related genes. The source of homologous genes:can be any species, e.g. primate species, particularly human; rodents, such as rats and mice; canines, felines, bovines, ovines, equines, yeast, nematodes, etc.

Preferably, hybridization is performed using at least 15 contiguous nucleotides (nt) of at least one of SEQ ID NOS:1-6010. That is, when at least 15 contiguous nt of one of the disclosed SEQ ID NOS. is used as a probe, the probe will preferentially hybridize with a nucleic acid comprising the complementary sequence, allowing the identification and retrieval of the nucleic acids that uniquely hybridize to the selected probe. Probes from more than one SEQ ID NO. can hybridize with the same nucleic acid if the cDNA from which they were derived corresponds to one mRNA. Probes of more than 15 nt can be used, e.g., probes of from about 18 nt to about 100 nt, but 15 nt represents sufficient sequence for unique identification.

The polynucleotides of the invention also include naturally occurring variants of the nucleotide sequences (e.g. degenerate variants, allelic variants, etc.). Variants of the polynucleotides of the invention are identified by hybridization of putative variants with nucleotide sequences disclosed herein, preferably by hybridization under stringent conditions. For example, by using appropriate wash conditions, variants of the polynucleotides of the invention can be identified where the allelic variant exhibits at most about 25-30% base pair (bp) mismatches relative to the selected polynucleotide probe. In general, allelic variants contain 15-25% bp mismatches, and can contain as little as even 5-15%, or 2-5%, or 1-2% bp mismatches, as well as a single bp mismatch.

The invention also encompasses homologs corresponding to the polynucleotides of SEQ ID NOS:1-6010, where the source of homologous genes can be any mammalian species, e.g., primate species, particularly human; rodents, such as rats; canines, felines, bovines, ovines, equines, yeast, nematodes, etc. Between mammalian species, e.g., human and mouse, homologs generally have substantial sequence similarity, e.g., at least 75% sequence identity, usually at least 90%, more usually at least 95% between nucleotide sequences. Sequence similarity is calculated based on a reference sequence, which may be a subset; of a larger sequence, such as a conserved motif, coding region, flanking region, etc. A reference sequence will usually be at least about 18 contiguous nt long, more usually at least about 30 nt long, and may extend to the complete sequence that is being compared. Algorithms for sequence analysis are known in the art, such as gapped BLAST, described in Altschul, et al. Nucleic Acids Res. (1997) 25:3389-3402.

In general, variants of the invention have a sequence identity greater than at least about 65%, preferably at least about 75%, more preferably at least about 85%, and can be greater than at least about 90% or more as determined by the Smith-Waterman homology search algorithm as implemented, in MPSRCH program (Oxford Molecular). For the purposes of this invention, a preferred method of calculating percent identity is the Smith-Waterman algorithm, using the following. Global DNA sequence identity must be greater than 65% as determined by the Smith-Waterman homology search algorithm as implemented in MPSRCH program (Oxford Molecular) using an affine gap search with the following search parameters gap open penalty, 12; and gap extension penalty, 1.

The subject nucleic acids can be cDNAs or genomic DNAs, as well as fragments thereof, particularly fragments that encode a biologically active gene product and/or are useful in the methods, disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

The nucleic acid compositions of the subject invention can encode all or a part of the subject polypeptides. Double or single stranded fragments can bc obtained from the DNA sequence by chemically synthesizing oligonucleotides in accordance with conventional methods, by restriction enzyme digestion, by PCR amplification, etc. Isolated polynucleotides and polynucleotide fragments of the invention comprise at least about 10, about 15, about 20, about 35, about 50, about 100, about 150 to about 200, about 250 to about 300, or about 350 contiguous nt selected from the polynucleotide sequences as shown in SEQ ID NOS:1-6010. For the most part, fragments will be of at least 15 nt, usually at least 18 nt or 25 nt, and up to at least about 50 contiguous nt in length or more. In a preferred embodiment, the polynucleotide molecules comprise a contiguous sequence of at least 12 nt selected from the group consisting of the polynucleotides shown in SEQ ID NOS: 1-6010.

Probes specific to the polynucleotides of the invention can be generated using the polynucleotide sequences disclosed in SEQ ID NOS: 1-6010. The probes are preferably at least about 12, 15, 16, 18, 20, 22, 24, or 25 nt fragment of a corresponding contiguous sequence or SEQ ID NOS:1-6010, and can be less than 2, 1, 0.5, 0.1, or 0.05 kb in length. The probes can be synthesized chemically or can be generated from longer polynucleotides using restriction enzymes. The probes can labeled, for example, with a radioactive, biotinylated, or fluorescent tag. Preferably, probes are designed based upon an identifying sequence of a polynucleotide of one of SEQ ID NOS:1-6010. More preferably, probes are designed based on a contiguous sequence of one of the subject polynucleotides that remain unmasked following application of a masking program for masking low complexity (e.g., XBLAST) to the sequence, i.e., one would select an unmasked region, as indicated by the polynucleotides outside the poly-n stretches of the masked sequence produced by the masking program.

The polynucleotides of the subject invention are isolated and obtained in substantial purity, generally as other than an intact chromosome. Usually, the polynucleotides, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome.

The polynucleotides of the invention can be provided as a linear molecule or within a circular molecule, and can be provided within autonomously replicating molecules (vectors) or within molecules without replication sequences. Expression of the polynucleotides can be regulated by their own or by other regulatory sequences known in the art. The polynucleotides of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA coated latex beads, protoplast fusion, viral infection, electroporation, gene gun, calcium phosphate-mediated transfection, and the like.

The subject nucleic acid compositions can be used to, for example, produce polypeptides, as probes for the detection of mRNA of the invention in biological samples (e.g., extracts of human cells) to generate additional copies of the polynucleotides, to generate ribozymes or antisense oligonucleotides, and as single stranded DNA probes or as triple-strand forming oligonucleotides. The probes described herein can be used to, for example, determine the presence or absence of the polynucleotide sequences as shown in SEQ ID NOS: 1-6010 or variants thereof in a sample. These and other uses are described in more detail below.

Use of Polynucleotides to Obtain Full-Length cDNA, Gene, and Promoter Region

In one embodiment, the polynucleotides are useful as starting materials to construct larger molecules. In one example, the polynucleotides of the invention are used to construct polynucleotides that encode a larger polypeptide (e.g., up to the full-length native polypeptide as well as fusion proteins comprising all or a portion of the native polypeptide) or may be used to produce haptens of the polypeptide (e.g., polypeptides useful to generate antibodies).

In one particular example, the polynucleotides of the invention are used to make or isolate cDNA molecules encoding all or portion of a naturally-occurring polypeptide. Full-length cDNA molecules comprising the disclosed polynucleotides are obtained as follows. A polynucleotide having a sequence of one of SEQ ID NOS:1-6010, or a portion thereof comprising at least 12, 15, 18, or 20 nt, is used as a hybridization probe to detect hybridizing members of a cDNA library using probe design methods, cloning methods, and clone selection techniques such as those described in U.S. Pat. No. 5,654,173. Libraries of cDNA are made from selected tissues, such as normal or tumor tissue, or from tissues of a mammal treated with, for example, a pharmaceutical agent. Preferably, the tissue is the same as the tissue from which the polynucleotides of the invention were isolated, as both the polynucleotides, described herein and the cDNA represent expressed genes. Most preferably, the cDNA library is made from the biological material described herein in the Examples. The choice of cell type for library construction can be made after the identity of the protein encoded by the gene corresponding to the polynucleotide of the invention is known. This will indicate which tissue and cell types are likely to express the related gene, and thus represent a suitable source for the mRNA for generating the cDNA. Where the provided polynucleotides are isolated from cDNA libraries, the libraries are prepared from mRNA of human colon cells, more preferably, human colon cancer cells, even more preferably, from a highly metastatic colon cell, Km12L4-A.

Techniques for producing and probing nucleic acid sequence libraries are described, for example, in Sambrook et al., Molecular Cloning: A Laboratory Manual, 2nd Ed., (1989) Cold Spring Harbor Press, Cold Spring Harbor, N.Y. The cDNA can be prepared by using primers based on polynucleotides comprising a sequence of SEQ ID NOS:1-6010. In one embodiment, the cDNA library can be made from only poly-adenylated mRNA. Thus, poly-T primers can be used to prepare cDNA from the mRNA.

Members of the library that are larger than the provided polynucleotides, and preferably that encompass the complete coding sequence of the native message, are obtained. In order to confirm that the entire cDNA has been obtained, RNA protection experiments are performed as follows. Hybridization of α-full-length cDNA to an mRNA will protect the RNA from RNase degradation. If, the cDNA is not full length then the portions of the mRNA that are not hybridized will be subject to RNase degradation. This is assayed, as is known in the art, by changes in electrophoretic mobility on polyacrylamide gels, or by detection of released monoribonucleotides. Sambrook et al., Molecular Cloning: A Laboratory Manual, 2nd Ed., (1989) Cold Spring Harbor Press, Cold Spring Harbor, N.Y. In order to obtain additional sequences 5′ to the end of a partial cDNA, 5′ RACE (PCR Protocols: A Guide to Methods and Applications, (1990) Academic Press, Inc.) can be performed.

Genomic DNA is isolated using the provided polynucleotides in a manner similar to the isolation of full-length cDNAs. Briefly, the provided polynucleotides, or portions thereof, are used as probes to libraries of genomic DNA. Preferably, the library is obtained from the cell type that was used to generate the polynucleotides of the invention, but this is not essential. Most preferably, the genomic DNA is obtained from the biological material described herein in the Examples. Such libraries can be in vectors suitable for carrying large segments of a genome, such as PI or YAC, as described in detail in Sambrook et al., supra, 9.4-9.30. In addition, genomic sequences can be isolated from human BAC libraries, which are commercially available from Research Genetics, Inc., Huntsville, Ala., USA, for example. In order to obtain additional 5′ or 3′ sequences; chromosome walking is performed, as described in Sambrook et al., such that adjacent and overlapping fragments of genomic DNA are isolated. These are mapped and pieced together, as is known in the art, using restriction digestion enzymes and DNA ligase.

Using the polynucleotide sequences of the invention, corresponding full-length genes can be isolated using both classical and PCR methods to construct and probe cDNA libraries. Using either method, Northern blots, preferably, are performed on a number of cell types to determine which cell, lines express the gene of interest at the highest level. Classical methods of constructing cDNA libraries are taught in Sambrook et al., supra. With these methods, cDNA can be produced from mRNA and inserted into viral or expression vectors. Typically, libraries of mRNA comprising poly(A) tails can be produced with poly(T) primers. Similarly, cDNA libraries can be produced using the instant sequences as primers.

PCR methods are used to amplify the members of a cDNA library that comprise the desired insert. In this case, the desired insert will contain sequence from the full length cDNA that corresponds to the instant polynucleotides. Such PCR methods include gene trapping and RACE methods. Gene trapping entails inserting a member of a cDNA library into a vector. The vector then is denatured to produce single stranded molecules. Next, a substrate-bound probe, such as a biotinylated oligo, is used to trap cDNA inserts of interest. Biotinylated probes can be linked to an avidin-bound solid substrate. PCR methods can be used to amplify the trapped cDNA. To trap sequences corresponding to the full length genes, the labeled probe sequence is based on the polynucleotide sequences of the invention. Random primers or primers specific to the library vector can be used to amplify the trapped cDNA. Such gene tapping techniques are described in Gruber et al., WO 95/04745 and Gruber et al., U.S. Pat. No. 5,500,356. Kits are commercially available to perform gene trapping experiments from for example, Life Technologies, Gaithersburg, Md., USA.

“Rapid amplification of cDNA ends,” or RACE, is a PCR method of amplifying cDNAs from a number of different RNAs. The cDNAs are ligated to an oligonucleotide linker, and amplified by PCR using two primers. One primer is based on sequence from the instant polynucleotides, for which full length sequence is desired, and a second primer comprises sequence that hybridizes to the oligonucleotide linker to amplify the cDNA A description of this method is reported in WO 97/19110. In preferred embodiments of RACE, a common primer is designed to anneal to an arbitrary adaptor sequence ligated to cDNA ends (Apte and Siebert, Biotechniques (1993) 15:890-893; Edwards et al., Nuc. Acids Res. (1991) 19:5227-5232). When a single gene-specific RACE primer is paired with the common primer, preferential amplification of sequences between the single gene specific primer and the common primer occurs. Commercial cDNA pools modified for use in RACE are available.

Another PCR-based method generates full-length cDNA library with anchored ends without needing specific knowledge of the cDNA sequence. The method uses lock-docking primers (I-VI), where one primer, poly TV (I-II) locks over the polyA tail of eukaryotic mRNA producing first strand synthesis and a second primer, polyA (IV-VI) locks onto the polyC tail added by terminal deoxynucleotidyl transferase (TdT)(see, e.g., WO 96/40998).

The promoter region of a gene generally is located 5′ to the initiation site for RNA polymerase II. Hundreds of promoter regions contain the “TATA” box, a sequence such as TATTA or TATAA, which is sensitive to mutations. The promoter region can be obtained by performing 5′ RACE using a primer from the coding region of the gene. Alternatively, the cDNA can be used as a probe for the genomic sequence, and the region 5′ to the coding region is identified by “walking up.” If the gene is highly expressed or differentially expressed, the promoter from the gene can be of use in a regulatory construct for a heterologous gene.

Once the full-length cDNA or gene is obtained, DNA encoding variants can be prepared by site-directed mutagenesis, described in detail in Sambrook et al., 15.3-15.63. The choice of codon or nucleotide to be replaced can be based on disclosure herein on optional changes in amino acids to achieve altered protein structure and/or function.

As an alternative method to obtaining DNA or RNA from a biological material, nucleic acid comprising nucleotides having the sequence of one or more polynucleotides of the invention can be synthesized, Thus, the invention encompasses nucleic acid molecules ranging in length from 15 nt (corresponding to at least 15 contiguous nt of one of SEQ ID NOS: 1-6010) up to a maximum length suitable for one or more biological manipulations, including replication and expression, of the nucleic acid molecule. The invention includes but is not limited to (a) nucleic acid having the size of a full gene and comprising at least one of SEQ ID NOS: 1-6010; (b) the nucleic acid of (a) also comprising at least one additional gene, operably linked to permit expression of a fusion protein; (c) an expression vector comprising (a) or (b); (d) a plasmid comprising (a) or (b); and (e) a recombinant viral particle comprising (a) or (b). Once provided with the polynucleotides disclosed herein, construction or preparation of (a)-(e) are well within the skill in the art.

The sequence of a nucleic acid comprising at least 15 contiguous nt of at least any one of SEQ ID NOS:1-6010, preferably the entire sequence of at least anyone of SEQ NOS:1-6010, is not limited and can be any sequence of A, T, G, and/or C (for DNA) and A, U, G, and/or C (for RNA) or modified bases thereof, including inosine and pseudouridine. The choice of sequence will depend on the desired function and can be dictated by coding regions desired, the intron-like regions desired, and the regulatory regions desired. Where the entire sequence of any one of SEQ ID NOS:1-6010 is within the nucleic acid, the nucleic acid obtained is referred to herein as a polynucleotide comprising the sequence of any one of SEQ ID NOS:1-6010.

Expression of Polypeptide Encoded by Full-Length cDNA or Full-Length Gene

The provided polynucleotides (e.g., a polynucleotide having a sequence of one of SEQ ID, NOS:1-6010), the corresponding cDNA, or the full-length gene is used to express a partial or complete gene product. Constructs of polynucleotides having sequences of SEQ ID NOS:1-6010 can also be generated synthetically. Alternatively, single-step assembly of a gene and entire plasmid from large numbers of oligodeoxyribonucleotides is described by, e.g., Stemmer et al., Gene (Amsterdam) (1995) 164(1):49-53. In this method, assembly PCR (the synthesis of long DNA sequences from large numbers of oligodeoxyribonucleotides (oligos)) is described. The method is derived from DNA shuffling (Stemmer, Nature (1994) 370:389-391), and does not rely on DNA ligase; but instead relies on DNA polymerase to build increasingly long DNA fragments during the assembly process.

Appropriate polynucleotide constructs are purified using standard recombinant DNA techniques, as described in, for example, Sambrook et al., Molecular Cloning: A Laboratory Manual, 2nd Ed., (1989) Cold Springs Harbor Press, Cold Spring Harbor, N.Y., and under current regulations described in United States Dept. of HHS, National Institute of Health (NIH) Guidelines for Recombinant DNA Research. The gee product encoded by a polynucleotide of the invention expressed in any expression system, including, for example, bacterial, yeast; insect, amphibian and mammalian systems. Vectors, host cells and methods for obtaining expression same are well known in the art. Suitable vectors and host cells are described in U.S. Pat. No. 5,654,173.

Polynucleotide molecule, comprising a polynucleotide sequence provided herein are generally propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. Methods for preparation of vectors comprising a desired sequence are well known in the art.

The polynucleotides set forth in SEQ ID NOS: 1-6010 or their corresponding full-length polynucleotides are linked to regulator sequences as appropriate to obtain the desired expression properties. These can include promoters (attached either at the 5′ end of the sense strand or at the 3′ end of the antisense strand) enhancers, terminators, operators, repressors, and inducers. The promoters can be regulated or constitutive. In some situations it may be desirable to use conditionally active promotes such as tissue-specific or developmental stage-specific promoters. Theses are linked to the desired nucleotide sequence using the techniques described above for linkage to vectors. Any techniques known in the art can be used.

When any of the above host cells, or other appropriate host cells or organisms, are used to replicates and/or express the polynucleotides or nucleic, acids of the invention, the resulting replicated nucleic acid, RNA, expressed protein or polypeptide, is within the scope of the invention as a product of the host cell or organism. The product is recovered by any appropriate means known in the art.

Once the gene corresponding to a selected polynucleotide is identified expression can be regulated in the cell to which the gene is native. For example, an endogenous gene of a cell can be, regulated by an exogenous regulatory sequence as disclosed in U.S. Pat. No. 5,641,670.

Identification of Functional and Structural Motifs

Translations of the nucleotide sequence of the provided polynucleotides, cDNAs or full genes can be aligned with individual known sequences. Similarity with individual sequences can be used to determine the activity of the polypeptides encoded by the polynucleotides of the invention. Also sequences exhibiting similarity with more than one individual sequence can exhibit activities that are characteristic of either or both individual sequences.

The full length sequences and fragments of the polynucleotide sequences of the nearest neighbors can be used as probes and primers to identify and isolate the full length sequence corresponding to provided polynucleotides. The nearest neighbors can indicate a tissue or cell type to be used to construct a library for the full-length sequences corresponding to the provided polynucleotides.

Typically, a selected polynucleotide is translated in all six frames to determine the best alignment with the individual sequences. The sequences disclosed herein in the Sequence Listing are in a 5′ to 3′ orientation and translation in three frames can be sufficient (with a few specific exceptions as described in the Examples). These amino acid sequences are referred to, generally, as query sequences, which will be aligned with the individual sequences. Databases with individual sequences are described in “Computer Methods for Macromolecular Sequence Analysis” Methods in Enzymology (1996) 266, Doolittle; Academic Press, Inc., a division of Harcourt Brace & Co., San Diego, Calif., USA. Databases include GenBank, EMBL, and DNA Database of Japan (DDBJ).

Query and individual sequences' can be aligned using the methods and computer programs described above, and include BLAST 2.0, available over the world wide web at a site supported by the National Center for Biotechnology Information, which is supported by the National Library of Medicine and the National Institutes of Health See also Altschul, et al. Nucleic Acids Res. (1997) 25:3389-3402. Another alignment algorithm is Fasta, available in the Genetics Computing Group (GCG) package, Madison, Wis., USA, a wholly owned subsidiary of Oxford Molecular Group, Inc. Other techniques for alignment are described in Doolittle, supra. Preferably, an alignment a program that permits gaps in the sequence is utilized to align the sequences. The Smith-Waterman is one type of algorithm that permits gaps in sequence alignments. See Meth. Mol. Biol. (1997) 70: 173-187. Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to identify sequences that are distantly related matches, and is especially tolerant of small gaps and nucleotide sequence errors. Amino acid sequences encoded by the provided polynucleotides can be used to search both protein and DNA databases. Incorporated; herein by reference are all sequences that have been made public as of the filing date of this application by any of the DNA or protein sequence databases, including the patent databases (e.g., GeneSeq). Also incorporated by reference are those sequences that have been submitted to these databases as of the filing date of the present application but not made public until after the filing date of the present application.

Results of individual and query sequence alignments can be divided into three categories: high similarity, weak similarity, and no similarity Individual alignment results ranging from high similarity to weak similarity provide a basis for determining polypeptide activity and/or structure. Parameters for categorizing individual results include: percentage of the alignment region length where the strongest alignment is found, percent sequence identity, and p value. The percentage of the alignment region length is calculated by counting the number of residues of the individual sequence found in the region of strongest alignment, e.g., contiguous region of the individual sequence that contains the greatest number of residues that are identical to the residues of the corresponding region of the aligned query sequence. This number is divided by the total residue length of the query sequence to calculate a percentage. For example, a query sequence of 20 amino acid residues might be aligned with a 20 amino acid region of an individual sequence. The individual sequence might be identical to amino acid residues 5, 9-15, and 17-19 of the query sequence. The region of strongest alignment is thus the region stretching from residue 9-19, an 11 amino acid stretch. The percentage of the alignment region length is 11 (length of the region of strongest alignment) divided by (query sequence length) 20 or 55%.

Percent sequence identity is calculated by counting the number of amino acid matches between the query and individual sequence and dividing total number of matches by the number of residues of the individual sequences found in the region of strongest alignment. Thus, the percent identity in the example above would be 10 matches divided by 11 amino acids, or approximately, 90.9%

P value is the probability that the alignment was produced by chance. For a single alignment, the p value can be calculated according to Karlin et al., Proc. Natl. Acad. Sci. (1990) 87:2264 and Karlin et al., Proc. Natl. Acad. Sci. (1.993) 90. The p value of multiple alignments using the same query sequence can be calculated using an heuristic approach described in Altschul et al., Nat. Genet. (1994) 6:119. Alignment programs such as BLAST program can calculate the p value. See also Altschul et al., Nucleic Acids Res. (1997) 25:3389-3402.

Another factor to consider for determining identity or similarity is the location of the similarity or identity. Strong local alignment cant indicate similarity even if the length of alignment is short. Sequence identity scattered throughout the length of the query sequence also can indicate a similarity between the query and profile sequences. The boundaries of the region where the sequences align can be determined according to Doolittle supra BLAST 2.0 (see, e.g., Altschul, et al. Nucleic Acids Res., (1997) 25:3389-3402) or FAST programs; or by determining the area where sequence identity is highest.

High Similarity. In general, in alignment results considered to be of high similarity, the percent of the alignment region length is typically at least about 55% of total length query sequence, more typically, at least about 58% even more typically; at least about 60% of the total residue length of the query sequence. Usually, percent length of the alignment region can be as much as about 62%; more usually, as much as about 64%; even more usually, as much as about 66%. Further, for high similarity, the region of alignment, typically, exhibits at least about 75% of sequence identity; more typically, at least about 78%; even more typically; at least about 80% sequence identity. Usually, percent sequence identity can be as much as about 82%; more usually, as much as about 84%; even more usually, as much as about 86%.

The p value is used in conjunction with these methods. If high similarity is found, the query sequence is considered to have high similarity with a profile sequence when the p value is less than or equal to about 10e-2; more usually; less than or equal to about 10e-3; even more usually; less than or equal to about 1e-4. More typically, the p value is no more than about 10e-5; more typically; no more than or equal to about 10e-10; even more typically, no more than or equal to about 10e-15 for the query sequence to be considered high similarity.

Weak Similarity. In general, where alignment results considered to be of weak similarity, there is no minimum percent length of the alignment region nor minimum length of alignment. A better showing of weak similarity is considered when the region of alignment is, typically, at least about 15 amino acid residues in length; more typically, at least about 20; even more typically, at least about 25 amino acid residues in length. Usually, length of the alignment region can be as much as about 30 amino acid residues, more usually, as much as about 40; even more usually, as much as about 60 amino acid residues. Further, for weak similarity, the region of alignment, typically, exhibits at least about 35% of sequence identity; more typically, at least about 40%; even more typically, at least about 45% sequence identity. Usually, percent sequence identity can be as much as about 50%; more profile sequence when the p value is usually less than or equal to about 10e-2; more usually, less than or equal to about 10e-3; even more usually; less than or equal to about 10e-4. More typically, the p value is no more than about 10e-5; more usually; no more than or equal to about 10e-10; even more usually, no more than or equal to about 10e-15 for the query sequence to be considered weak similarity.

Similarity Determined by Sequence Identity Alone. Sequence identity alone can be used to determine similarity of a query sequence to an individual sequence and can indicate the activity of the sequence. Such an alignment, preferably, permits gaps to align sequences. Typically, the query sequence is related to the profile sequence if the sequence identity over the entire query sequence is at least about 15%; more typically, at least about 20%; even more typically, at least about 25%; even more typically, at least about 50%. Sequence identity alone as a measure of similarity is most useful when the query sequence is usually, at least 80 residues in length; more usually, at least 90 residues in length; even more usually, at least 95 amino acid residues in length. More typically, similarity can be concluded based on sequence identity alone when the query sequence is preferably 100 residues in length; more preferably, 120 residues in length; even more preferably, 150 amino acid residues in length.

Alignments with Profile and Multi 1e Aligned Sequences. Translations of the provided polynucleotides can be aligned with amino acid profiles that define either protein families or common motifs. Also, translations of the provided polynucleotides can be aligned to multiple sequence alignments (MSA) comprising the polypeptide sequences of members of protein families or motifs. Similarity or identity with profile sequences or MSAs can be used to determine the activity of the gene products (e.g., polypeptides) encoded by the provided polynucleotides or corresponding cDNA or genes. For example, sequences that show an identity or similarity with a chemokine profile or MSA can exhibit chemokine activities.

Profiles can be designed manually by (1) creating an MSA, which is an alignment of the amino acid sequence of members that belong to the family and (2) constructing a statistical representation of the alignment. Such methods are described, for example, in Birney et al., Nucl. Acid Res. (1996) 24(14): 2730-2739. MSAs of some protein families and motifs are publicly available. For example the Genome Sequencing Center at the Washington University School of Medicine provides a web set (Pfam) which provides MSAs of 547 different families and motifs. These MSAs are described also in Sonnhammer et al., Proteins (1997) 28: 405-420. Other sources over the world wide web include the site supported by the European Molecular Biology Laboratories in Heidelberg, Germany. A brief description of these MSAs is reported in Pascarella et al., Prot. Eng. (1996) 9(3):249-251. Techniques for building profiles from MSAs are described in Sonnhammer et al., supra; Birney et al., supra; and “Computer Methods for Macromolecular Sequence Analysis,” Methods in Enzymology (1996) 266, Doolittle, Academic Press, Inc, San Diego, Calif. USA.

Similarity between a query sequence and a protein family or motif can be determined by (a) comparing the query sequence against the profile and/or (b) aligning the query sequence with the members of the family or motif. Typically, a program such as Searchwise is used to compare the query sequence to the statistical representation of the multiple alignment, also known as a profile (see Birney et al., supra). Other techniques to compare the sequence and profile are described in Sonnhammer et al., supra and Doolittle, supra.

Next, methods described by Feng et al., J. Mol. Evol. (1987) 25:351 and Higgins et al., CABIOS (1989) 5:151 can be used align the query sequence with the members of a family or motif also known as a MSA. Sequence alignments can be generated using any of a variety of software tools. Examples include PileUp, which creates a multiple sequence alignment, and is described in Feng et al., J. Mol. Evol. (1987) 25:351. Another method, GAP, uses the alignment method of Needleman et al., J. Mol. Biol. (1970) 48:43. GAP is best suited for global alignment of sequences. A third method, BestFit, functions by inserting gaps to maximize the number of matches using the local homology algorithm of Smith et al., Adv. Appl. Math. (1981) 2:482. In general, the following factors are used to determine if a similarity between a query sequence and a profile or MSA exists: (1) number of conserved residues found in the query sequence, (2) percentage of conserved residues found in the query sequence, (3) number of frame shifts, and (4) spacing between conserved residues.

Some alignment programs that both translate and align sequences can make any number of frameshifts when translating the nucleotide sequence to produce the best alignment. The fewer frameshifts needed to produce an alignment, the stronger the similarity or identity between the query and profile or MSAs. For example, a weal, similarity resulting from no frameshiits can be a better indication of activity or structure of a query sequence, than a strong similarity resulting from two frameshifts. Preferably, three or fewer frameshifts are found in an alignment; more preferably two or fewer frameshifts; even more preferably, one or fewer frameshifts; even more preferably, no frameshifts are found in an alignment of query and profile or MSAs.

Conserved residues are those amino acids found at a particular position in all or some of the family or motif members. Alternatively, a position is considered conserved if only a certain class of amino acids is found in a particular position in all or some of the family members. For example, the N-terminal position can contain a positively charged amino, acid, such as lysine, arginine, or histidine.

Typically, a residue of a polypeptide is conserved when a class of amino acids or a single amino acid is found at a particular position in at least about 40% of all class members; more typically at least about 50%; even more typically, at least about 60% of the members. Usually, a residue is conserved when a class or single amino acid is found in at least about 70% of the members of a family or motif; more usually, at least about 80%; even more usually, at least about 95%.

A residue is considered conserved when three unrelated amino acids are found at a particular position in some or all of the members; more usually, two unrelated amino acids. These residues are conserved when the unrelated amino acids are found at particular positions in at least about 40% of all class member; more typically, at least about 50%, even more typically, at least about 60% of the members. Usually, a residue is conserved when a class or single amino acid is found in at least about 70% of the members of a family or motif, more usually, at least about 80%; even more usually, at least about 90%; even more usually, at least about 95%.

A query sequence has similarity to a profile or MSA when, the query sequence comprises at least about 25% of the conserved residues of the profile or MSA; more usually, at least about 30%; even more usually; at least about 40%. Typically the query sequence has a stronger similarity to a profile sequence or MSA when the query sequence comprises at least about 45% of the conserved residues of the profile or MS more typically, at least about 50%; even more typically, at least about 55%.

Identification of Secreted & Membrane-Bound Polypeptides. Both secreted and membrane-bound polypeptides of the present invention are of particular interest. For example, levels of secreted polypeptides can be assayed in body fluids that are convenient, such as blood, plasma, serum, and other body fluids such as urine, prostatic fluid and semen. Membrane-bound polypeptides are useful for constructing vaccine antigens or inducing an immune response. Such antigens would comprise all or part of the extracellular region of the membrane-bound polypeptides. Because both secreted and membrane-bound polypeptides comprise a fragment of contiguous hydrophobic amino acids, hydrophobicity predicting algorithms can be used to identify such polypeptides.

A signal sequence is usually encoded by both secreted and membrane-bound polypeptide genes to direct a polypeptide to the surface of the cell. The signal sequence usually comprises a stretch of hydrophobic residues. Such signal sequences can fold into helical structures. Membrane-bound polypeptides typically comprise at least one transmembrane region that possesses a stretch of hydrophobic amino acids that can transverse the membrane. Some transmembrane regions also exhibit a helical structure. Hydrophobic fragment within a polypeptide c an be identified by using computer algorithms. Such algorithms include Hopp & Woods, Proc. Natl. Acad. Sci. USA (1981) 78:3824-3828; Kyte & Doolittle, J. Mol. Biol. (1982) 157: 105-132; and RAOAR algorithm, Degli Esposti et al., Eur. J. Biochem. (1990) 19: 207-219.

Another method of identifying secreted and membrane-bound polypeptides is to translate the polynucleotides of the invention in all six frames and determine if at least 8 contiguous hydrophobic amino acids are present. Those translated polypeptides with at least 8; more typically, 10; even more typically, 12 contiguous hydrophobic amino acids are considered to be either a putative secreted or membrane bound polypeptide. Hydrophobic amino acids include alanine, glycine, histidine, isoleucine, leucine, lysine, methionine, phenylalanine, proline, threonine, tryptophan, tyrosine, and valine

Identification of the Function of an Expression Product of a Full-Length Gene

Ribozymes, antisense constructs, and dominant negative mutants can be used to determine function of the expression product of a gene corresponding to a polynucleotide provided herein. These methods and compositions are particularly useful where the provided novel polynucleotide exhibits no significant or substantial homology to a sequence encoding a gene known function. Antisense molecules and ribozymes can be constructed from synthetic polynucleotides. Typically, the phosphoramidite method of oligonucleotide synthesis is used. See Beaucage et al., Tet. Lett. (1981) 22:1859 and U.S. Pat. No. 4,668,777. Automated devices for synthesis are available to create oligonucleotides using this chemistry. Examples of such devices include Biosearch 8600, Models 392 and 394 by Applied Biosystems, a division of Perkin-Elmer Corp., Foster City, Calif., USA; and Expedite by Perceptive Biosystems, Framingham, Mass., USA. Synthetic RNA, phosphate analog oligonucleotides, and chemically derivatized oligonucleotides can also be produced, and can be covalently attached to other molecules. RNA oligonucleotides can be synthesized, for example, using RNA phosphoramidites. This method can be performed on an automated synthesizer, such as Applied Biosystems, Models 392 and 394, Foster City, Calif., USA.

Phosphorothioate oligonucleotides can also be synthesized for antisense construction. A sulfurizing reagent, such as tetraethylthiruam disulfide (TETD) in acetonitrile can be used to convert the internucleotide cyanoethyl phosphite to the phosphorothioate triester within 15 minutes at room temperature. TETD replaces the iodine reagent, while all other reagents used for standard phosphoramidite chemistry remain the same. Such a synthesis method can be automated using Models 392 and 394 by Applied Biosystems, for example.

Oligonucleotides of up to 200 nt can be synthesized, more typically, 100 nt more typically 50 nt; even more typically, 30 to 40 nt. These synthetic fragments can be annealed and ligated together to construct larger fragments. See, for example, Sambrook et al., supra. Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting the phenotypic effect. One commonly used ribozyme motif is the hammerhead, for which the substrate sequence requirements are minimal. Design of the hammerhead ribozyme, as well as therapeutic uses of ribozymes, are disclosed in Usman et al., Current Opin. Struct. Biol. (1996) 6:527. Methods for production of ribozymes, including hairpin structure ribozyme fragments, methods of increasing ribozyme specificity and the like are known in the art.

The hybridizing region of the ribozyme can be modified or can be prepares as a branched structure as descried in Horn and Urdea, Nucleic Acids Res. (1989) 17:699. The basic structure of the ribozymes can also be chemically altered in ways familiar to those skilled in the art, and chemical synthesized ribozymes can be administered as synthetic oligonucleotide derivatives modified by monomeric units. In a therapeutic context, liposome mediated delivery of ribozymes improves cellular uptake, as described in Birikh et al., Eur. J. Biochem. 1997) 245:1.

Antisense nucleic acids are designed to specifically bind to RNA, resulting in the formation of RNA-DNA or RNA-RNA hybrids, with an arrest of DNA replication, reverse transcription or messenger RNA translation. Antisense polynucleotides based on a selected polynucleotide sequence can interfere with expression of the corresponding gene. Antisense polynucleotides are typically generated within the cell by expression from antisense constructs that contain the antisense strand as the transcribed strand. Antisense polynucleotides based on the disclosed polynucleotides will bind and/or interfered with the translation of mRNA comprising a sequence complementary to the antisense polynucleotide. The expression products of control cells and cells treated with the antisense construct are compared to detect the protein product of the gene corresponding to the polynucleotide upon which the antisense construct is based. The protein is isolated and identified using routine biochemical methods.

Given, the extensive background literature and clinical experience in antisense therapy, one skilled in the art can use selected polynucleotides of the invention as additional potential therapeutics. The choice of polynucleotide can be narrowed by first testing them for binding to a “hot spot” regions of the genome of cancerous cells. If a polynucleotide is identified as binding to a “hot spot,” testing the polynucleotide as antisense compound in the corresponding cancer cells is warranted.

As an alternative method for identifying function of the gene corresponding to a polynucleotide disclosed herein, dominant negative mutations are readily generated for corresponding proteins that are active as homomultimers. A mutant polyeptide will interact with wild-type polypeptides (made from the other allele) and form a non-functional multimer. Thus, a mutation is in a substrate-binding domain, a catalytic domain, or a cellular localization domain. Preferably, the mutant polypeptide will be overproduced. Point mutations are made that have such an effect. In addition, fusion, of different polypeptides of various lengths to the terminus of a protein can yield dominant negative mutants. General strategies are available for making dominant negative mutants (see e.g., Herskowitz, Nature (1987) 329:219). Such techniques can be used to create loss of function mutations, which are useful for determining protein function.

Polypeptides and Variants Thereof

The polypeptides of the invention include those encoded by the disclosed polynucleotides, as well as nucleic acids that, by virtue of the degeneracy of the genetic code, are not identical in sequence to the disclosed polynucleotides. Thus, the invention includes within its scope a polypeptide encoded by a polynucleotide having the sequence of any one of SEQ ID NOS:1-6010 or a variant thereof.

In general, the term “polypeptide” as used herein refers to both the full length polypeptide encoded by the recited polynucleotide, the polypeptide encoded by the gene represented by the recited polynucleotide, as well as portions or fragments thereof. “Polypeptide” also includes variants of the naturally occurring proteins, where such variants are homologous or substantially similar to the naturally occurring protein, and can be of an origin of the same or different species as the naturally occurring protein (e.g., human, murine or some other species that naturally expresses the recited polypeptide, usually a mammalian species). In general, variant polypeptides have a sequence that has at least about 80%, usually at least about 90%, and more usually at least about 98% sequence identity with a differentially expressed polypeptide of the invention, as measured by BLAST 2.0 using the parameters described above. The variant polypeptides can be naturally or non-naturally glycosylated, i.e., the polypeptide has a glycosylation pattern that differs from the glycosylation pattern found in the corresponding naturally occurring protein.

The invention also encompasses homologs of the disclosed polypeptides (or fragments thereof) where the homologs are isolated from other species, i.e. other animal or plant species, where such homologs, usually mammalian species, e.g., rodents, such as mice, rats; domestic animals, e.g., horse, cow, dog, cat, and humans. By “homolog” is meant a polypeptide having at least about 35%, usually at least about 40% and more usually at least about 60% amino acid sequence identity to a particular differentially expressed protein as identified above, where sequence identity is determined using the BLAST 2.0 algorithm, with the parameters described supra.

In general, the polypeptides of the subject invention are provided in a non-naturally occurring environment, e.g. are separated from their naturally occurring environment. In certain embodiments, the subject protein is present in a composition that is enriched for the protein as compared to a control. As such, purified polypeptide is provided, where by purified is meant that the protein is present in a composition that is substantially free of non-differentially expressed polypeptides, where by substantially free is meant that less than 90%, usually less than 60% and more usually less than 50% of the composition is made up of non-differentially expressed polypeptides.

Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence). Selection of amino acid alterations for production of variants can be based upon accessibility (interior vs. exterior) of the amino acid (see, e.g., Go et al, Int. J. Peptide Protein Res. (1980) 15:211), the thermostability of the variant polypeptide (see, e.g., Querol et al., Prot. Eng. (1996) 9:265), desired glycosylation sites (see e.g., Olsen and Thomsen, J. Gen. Microbiol. (1991) 137:579), desired disulfide bridges (see, e.g., Clarke et al., Biochemistry (1993) 32:4322; and Wakarchuk et al., Protein Eng. (1994) 7:1379), desired metal binding sites (see, e.g., Toma et al., Biochemistry (1991) 30:97 and Haezerbrouck et al., Protein Eng. (11993) 6:643), and desired substitutions within proline lops (see, e.g., Masul et al., Appl. Env. Microbiol. (1994) 60:3579). Cysteine-depleted muteins can be produced as disclosed in U.S. Pat. No. 4,959,314.

Variants also include fragments of the polypeptides disclosed herein, particularly haptens, biologically active fragments, and/corresponding to functional domains. Fragments of interest will typically be at least about 10 aa to at least about 15 aa in length, usually at least about 50 aa in length, and can be as long as 300 aa in length or longer, but will usually not exceed about 1000 aa in length where the fragment will have a stretch of amino acids that is identical to a polypeptide encoded by a polyucleotide having a sequence of any SEQ ID NOS: 1-6010, or a homolog thereof. The protein variants described herein are encoded by polynucleotides that are within the scope of the invention. The genetic code can be used to select the appropriate codons to construct the corresponding variants.

Computer-Related Embodiments

In general, a library of polynucleotides is a collection of sequence information, which information is provided in either biochemical form (e.g., as a collection of polynucleotide molecules), or in electronic form (e.g., as a collection of polynucleotide sequences stored in a computer-readable form, as in a computer system and/or as part of a computer program). The sequence information of the polynucleotides can be used in a variety of ways, e.g., as a resource for gene discovery, as a representation of sequences expressed in a selected cell type (e.g., cell type markers), and/or as markers of a given disease or disease state. In general, a disease marker is a representation of a gene product that is present in all cells, affected by disease either at an increased or decreased level relative to a normal cell (e.g. a cell of the same or similar type that is not substantially affected by disease). For example, a polynucleotide sequence in a library can be, a polynucleotide that represents an mRNA, polypeptide, or other gene product encoded by the polynucleotide, that is either overexpressed or underexpressed in a breast ductal cell affected by cancer relative to a i.e., substantially free) breast cell.

The nucleotide sequence information of the library can be embodied in any suitable form, e.g., electronic or biochemical forms. For example, a library of sequence information embodied in electronic form comprises an accessible computer data file (or, in biochemical form, a collection of nucleic acid molecules) that contains the representative nucleotide sequences of genes that are differentially expressed (e.g., overexpressed or underexpressed) as between for example, i) a cancerous cell and a normal cell; ii) c cancerous cell and a dysplastic cell, iii) a cancerous cell and a cell affected by a disease or condition other than cancer; iv) a metastatic cancerous cell and a normal cell and/or non-metastatic cancerous cell; v) a malignant cancerous cell and a non-malignant cancerous cell (or a normal cell) and/or vi) a dysplastic cell relative to a normal cell. Other combinations and comparisons of cells affected by various diseases or stages of disease will be readily apparent to the ordinarily skilled artisan. Biochemical embodiments of the library include a collection of nucleic acids that have the sequences of the genes in the library, where the nucleic acids can correspond to the entire gene in the library or to a fragment thereof, as described in greater detail below.

The polynucleotide libraries of the subject invention generally comprise sequence information of a plurality of polynucleotide sequences, where at least one of the polynucleotides has a sequence of any of SEQ ID NOS:1-6010. By plurality is meant at least 2, usually at least 3 and can include up to all of SEQ ID NOS:1-6010. The length and number of polynucleotides in the library will vary with the nature of the library, e.g., if the library is an oligonucleotide array, a cDNA array, computer database of the sequence information, etc.

Where the library is an electronic library, the nucleic acid sequence information can be present in a variety of media. “Media” refers to a manufacture, other than an isolated nucleic acid molecule, that contains the sequence information of the present invention. Such a manufacture provides the genome sequence or a subset thereof in a form that can be examined by means not directly applicable to the sequence as it exists in a nucleic acid. For example, the nucleotide sequence of the present invention, e.g. the nucleic acid sequences of any of the polynucleotides of SEQ ID NOS:1-6010, can be recorded on computer readable media, e.g. any medium that can be read and accessed directly by a computer. Such media include, but are not limited to: magnetic storage media, such as a floppy disc, a hard disc storage medium, and a magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present sequence information. “Recorded” refers to a process for storing information on computer readable medium, using any such methods as known in the art. Any convenient data storage structure can be chosen, based on the means used to access the store d information. A variety of data processor programs and formats can be used for storage, e.g. word processing text file, database format, etc. In addition to the sequence information, electronic versions of the libraries of the invention can be provided in conjunction or connection with other computer-readable information and/or other types of computer-readable files (e.g., searchable files, executable files, etc, including, but not limited to, for example, search program software, etc.).

By providing the nucleotide sequence in computer readable firm, the information can be accessed for a variety of purposes. Computer software to access sequence information is publicly available. For example, the gapped BLAST (Altschul et al. Nucleic Acids Res. (1997) 25:3389-3402) and BLAZED (Brutlag et al. Comp. Chem. (1993) 17:203) search algorithms on a Sybase system can be used to identify open reading frames (ORFs) within the genome that contain homology to ORFs from other organisms.

As used herein, “a computer-based system” refers to the hardware means, software means, and data storage means used to analyze the nucleotide sequence information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means can comprise any manufacture comprising a recording of the present sequence information as described above, or a memory access means that can access such a manufacture.

“Search means” refers to one or more programs implemented on the computer-based system to compare a target sequence or target structural motif, or expression levels of a polynucleotide in a sample, with the stored sequence information. Search means can be used to identify fragments or regions of the genome that match a particular target sequence or target motifs A variety of known algorithms are publicly known and commercially available, e.g. MacPattern (EMBL), BLASTN and BLASTX (NCBI). A “target sequence” can be any polynucleotide or amino acid sequence of six or more contiguous nucleotides or two or more amino acids, preferably from about 10 to 100 amino acids or from about 30 to 300 nt. A variety of comparing means can be used to accomplish comparison of sequence information from a sample (e.g., to analyze target sequences, target motifs, or relative expression levels) with the data storage means. A skilled artisan can readily recognize that any one of the publicly available homology search programs can be used as the search means for the computer based systems of the present invention to accomplish comparison of target sequences and motifs. Computer programs to analyze expression levels in a sample and in controls are also known in the art.

A “target structural motif,” “target motif,” refers to any rationally selected sequence or combination of sequences in which the sequence(s) are chosen based on a three-dimensional configuration that is formed upon the folding of the target motif, or on consensus sequences of regulatory or active sites. There are a variety of target motifs known in the art. Protein target motifs include, but are not limited to, enzyme active sites and signal sequences. Nucleic acid target motifs include, but are not limited to, hairpin structures, promoter sequences and other expression elements such as binding sites for transcription factors.

A variety of structural formats for the input and output means can be used to input and output the information in the computer-based systems of the present invention. One format for an output means ranks the relative expression levels of different polynucleotides. Such presentation provides a skilled artisan with a ranking of relative expression levels to determine a gene expression profile.

As discussed above, the “library” of the invention also encompasses biochemical libraries of the polynucleotides of SEQ ID NOS: 1-6010, e.g., collections of nucleic acids representing the provided polynucleotides. The biochemical libraries can take a variety of forms, e.g., a solution of cDNAs, a pattern of probe nucleic acids stably associated with a surface of a solid support (i.e., an array) and the like. Of particular interest are nucleic acid arrays in which one or more of SEQ ID NOS: 1-6010 is represented on the array. By array is meant a an article of manufacture that has at least a substrate with at least two distinct nucleic acid targets on one of its surfaces, where the number of distinct nucleic acids can be considerably higher, typically being at least 10, usually at least 20, and often at least 25 distinct nucleic acid molecules. A variety of different array formats have been developed and are known to those of skill in the art. The arrays of the subject invention find use in a variety of applications, including gene expression analysis, drug screening, mutation analysis and the like, as disclosed in the above-listed exemplary patent documents.

In addition to the above nucleic acid libraries, analogous libraries of polypeptides are also provided, where the polypeptides of the library will represent at least a portion of the polypeptides encoded by a gene corresponding to one or more of SEQ ID NOS 1′-6010.

Utilities

The polynucleotides of the invention are useful in a variety of applications. Exemplary, utilities of the polynucleotides of the invention are described below.

Construction of Larger Molecules: Recombinant DNAs and Nucleic Acid Multimers. In one embodiment of particular interest the polyucleotides described herein as, useful as the building blocks for larger molecules. In one example, the polynucleotide is a combonent of a larger cDNA molecule which in turn can, be adapted for expression in a host cell (e.g., a bacterial or eukaryotic (e.g. yeast or mammalian) host cell). The cDNA can include, in addition to the polypeptide encoded by the starting material polynucleotide (i.e., a polynucleotide described herein), an amino acid sequence that is heterologous to the polypeptide encoded by the polynucleotide described herein (e.g. as in a sequence encoding a fusion protein. In some embodiments, the polynucleotides described herein is used as starting; material polynucleotide for synthesizing all or a portion of the gene to which the described polynucleotide corresponds. For example, a DNA molecule encoding a full-length human polypeptide can be constructed using a polynucleotide described herein as starting material.

In another embodiment, the polynucleotides of the invention are used in nucleic acid multimers Nucleic acid multimers can be linear or branched polymers of the same repeating single-stranded oligonucleotide unit or different single-stranded oligonucleotide-units. Where the molecules are branched, the multimers are generally described as either “fork” or “comb” structures. The oligonucleotide units of the multimer may be composed of RNA, DNA modified nucleotides or combinations thereof. At least one of the units has a sequence, length, and composition that permits it to bind specifically to a first single-stranded nucleotide sequence of interest, typically analyte or an oligonucleotide bound to the analyte. In order to achieve such specificity and stability, this unit will normally be 15 to 50 nt, preferably −15 to 30 nt, in length and have a GC content in the range of 40% to 60%. In addition to such unit(s), the multimer includes a multiplicity of units that are capable of hybridizing specifically and stably to a second single-stranded nucleotide of interest, typically a labeled oligonucleotide or another multimer. These units will also normally be 15 to 50 nt, preferably 15 to 30 nt, in length and have a GC content in the range of 40% to 60%. When a multimer is designed to be hybridized to another multimer, the first and second oligonucleotide units are heterogeneous (different). One or more of the polynucleotides described herein, or a portion of a polynucleotide described herein, can be used as a repeating unit of such nucleic acid multimers.

The total number of oligonucleotide units in the multimer will usually be in the range of 3 to 50, more usually 10 to 20. In multimers in which the unit that hybridizes to the nucleotide sequence of interest is different from the unit that hybidizes to the labeled oligonucleotide, the number ratio of the latter to the former will usually be 2:1 to 30:1, more usually 5:1 to 20:1, and preferably 10:1 to 15:1.

The oligonucleotide unit the multimer may be covalently linked directly to each other through phosphodiester bonds or through interposed linking agents such as nucleic acid, amino acid, carbohydrate or polyol bridges, or through other cross-linking agents that are capable of cross-linking nucleic acid or modified nucleic acid strands. The site(s) of linkage may be at the ends of the unit (in either normal 3,-5′ orientation or randomly oriented) and/or at one or more internal nucleotides in the strand. In linear multimers the individual units are linked end-to-end to form a linear polymer. In one type of branched multimer three or more oligonucleotide units emanate from a pint of origin to form a branched structure. The point of origin may be another oligonucleotide unit or a multifunctional molecule to which at least three units can be covalently bound. In another type, there is an oligonucleotide unit backbone with one or more pendant oligonucleotide units. These latter-type multimers are “fork-like”, “comb-like” or combination “fork-” and “comb-like” in structure. The pendant units will normally depend from a modified nucleotide or other organic moiety having appropriate functional groups to which oligonucleotides may be conjugated or otherwise attached. The multimer may be totally linear, totally branched, or a combination of linear and branched portions preferably there will be at least two branch points in the multimer, mere preferably at least 3, preferably 5 to 10. The multimer may include one or more segments of double-stranded sequences.

Multimeric nucleic acid molecules are useful in amplifying the signal that results from a hybridization of one the first sequence of the multimeric molecule to a target sequence. The amplification is theoretically proportional to the number of iterations of the second segment.

Without being held to theory, forked structures of greater than about eight branches exhibited steric hindrance which inhibited binding of labeled probes to the multimer. On the other hand, comb structures exhibit little or no steric problems and are thus a preferred type of branched multimer. For a description of branched nucleic acid multimers of both the fork and comb types, as well as methods of use and synthesis, see e.g. U.S. Pat. Nos. 5,124,246 (fork-type structures); 5,710,264 (synthesis of comb structures); and 5,849,481.

Use of Polynucleotide Probes in Mapping and in Tissue Profiling. Polynucleotide probes, generally comprising at least 12 contiguous nt of a polynucleotide as shown in the Sequence Listing, are used for a variety of purposes, such as chromosome mapping of the polynucleotide and detection of transcription levels. Additional disclosure about preferred regions of the disclosed polynucleotide sequences is found in the Examples. A probe that hybridizes specifically to a polynucleotide disclosed herein should provide a detection signal at least 5-, 10-, or 20-fold higher than the background hybridization provided with other unrelated sequences.

Detection of Expression Levels. Nucleotide probes are used to detect expression of a gene corresponding to the provided polynucleotide. In Northern blots, mRNA is separated electrophoretically and contacted with a probe. A probe is detected as hybridizing to an mRNA species of a particular size. The amount of hybridization is quantitated to determine relative amounts of expression, for example under a particular condition. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluors, and enzymes. Other examples of nucleotide hybridization assays are described in WO92/02526 and U.S. Pat. No. 5,124,246.

Alternatively, the Polymerase Chain Reaction (PCR) is another means for detecting small amounts of target nucleic acids (see, e.g., Mullis et al., Meth. Enzymol. (1987) 155:335; U.S. Pat. No. 4,683,195; and U.S. Pat. No. 4,683,202). Two primer polynucleotides nucleotides that hybridize with the target nucleic acids are used to prime the reaction. The primers can be composed of sequence within or 3′ and 5′ to the polynucleotides of the Sequence Listing. Alternatively, if the primers are 3′ and 5′ to these polynucleotides, they need not hybridize to them or the complements. After amplification of the target with a thermsostable polymerase, the amplified target nucleic acids can be detected by methods known in the art, e.g., Southern blot. mRNA or cDNA can also be detected by traditional blotting techniques (e.g., Southern blot, Northern blot, etc.) described in Sambrook et al., “Molecular Cloning: A Laboratory Manual” (New York, Cold Spring Harbor Laboratory, 1989) (e.g., without PCR amplification). In general, mRNA or cDNA generated from mRNA using a polymerase enzyme can be purified and separated using gel electrophoresis, and transferred to a solid support, such as nitrocellulose. The solid support is exposed to a labeled probe, washed to remove any unhybridized probe, and duplexes containing the labeled probe are detected.

Mapping Polynucleotides of the present invention can be used to identify a chromosome on which the corresponding gene resides. Such mapping can be useful in identifying the function of the polynucleotide-related gene by its proximity to other genes with known function. Function can also be assigned to the polynucleotide-related gene when particular syndromes or diseases map to the same chromosome. For example, use of polynucleotide probes in identification and quantification of nucleic acid sequence aberrations is described in U.S. Pat. No. 5,783,387. An exemplary mapping method is fluorescence in situ hybridization (FISH), which facilitates comparative genomic hybridization to allow total genome assessment of changes in relative copy number of DNA sequences (see, e.g., Valdes et al., Methods in Molecular Biology (1997) 68:1). Polynucleotides can also be mapped to particular chromosomes using, for example, radiation hybrids or chromosome-specific hybrid panels. See Leach et al., Advances in Genetics, (1995) 33:63-99; Walter et al., Nature Genetics (1994) 7:22; Walter and, Goodfellow, Trends in Genetics (1992) 9:352. Panels for radiation hybrid mapping are available from Research Genetics, Inc., Huntsville, Ala., USA. Databases for markers using various panels are available via the world wide web at, sites supported by the Stanford Human Genome Center (Stanford University) and the Whitehead Institute for Biomedical Research/MIT Center for Genome Research. The statistical program RHMAP can be used to construct a map based on the data from radiation hybridization with a measure of the relative likelihood of one order versus another. RHMAP is available via the world wide web at a site supported by the University of Michigan. In addition, commercial programs are available for identifying regions of chromosomes commonly associated with disease, such as cancer.

Tissue Typing of Profiling. Expression of specific mRNA corresponding to the provided polynucleotides can vary in different cell types and can be tissue-specific. This variation of mRNA levels in different cell types can be exploited with nucleic acid probe assays to determine tissue types. For example, PCR, branched DNA probe assays, or blotting techniques utilizing nucleic acid probes substantially identical or complementary to polynucleotides listed in the Sequence Listing can determine the presence or absence of the corresponding cDNA or mRNA.

Tissue typing can be used to identify the developmental organ or tissue source of a metastatic lesion by identifying the expression of a particular marker of that organ or tissue. If a polynucleotide is expressed only in a specific tissue type, and a metastatic lesion is found to express that polynucleotide, then the developmental source of the lesion has been identified. Expression of a particular polynucleotide can be assayed by detection of either the corresponding mRNA or the protein product. As would be readily apparent to any forensic scientist, the sequences disclosed herein are useful in differentiating human tissue from non-human tissue. In particular, these sequences are useful to differentiate human tissue from bird, reptile, and amphibian tissue, for example.

Use of Polymorphisms. A polynucleotide of the invention can be used in forensics, genetic analysis, mapping, and diagnostic applications where the corresponding region of a gene is polymorphic in the human population. Any means for detecting a polymorphism in a gene can be used, including, but not limited to electrophoresis of protein polymorphic variants, differential sensitivity to restriction enzyme cleavage, and hybridization to allele-specific probes.

Antibody Production. Expression products of a polynucleotide of the invention as well as the corresponding, mRNA, cDNA, or complete gene, can be prepared and used for raising antibodies for experimental, diagnostic, and therapeutic purposes. For polynucleotides to which a corresponding gene has not been assigned, this provides an additional method of identifying the corresponding gene. The polynucleotide or related cDNA is expressed as described above, and antibodies are prepared. These antibodies are specific to an epitope on the polypeptide encoded by the polynucleotide, and can precipitate or bind to the corresponding native protein in a cell or tissue preparation or in a cell-free extract of an in vitro expression system.

Methods for production of antibodies that specifically bind a selected antigen are well known in the art. Immunogens, for, raising antibodies can be prepared by mixing a polypeptide encoded by a polynucleotide of the invention with an adjuvant, and/or by making fusion proteins with larger immunogenic proteins. Polypeptides can also b& covalently linked to other larger immunogenic proteins, such as keyhole limpet hemocyanin. Immunogens are typically administered intradermally, subcutaneously, or intramuscularly to experimental animals such as rabbits, sheep), and mice, to generate antibodies. Monoclonal antibodies can be generated by isolating spleen cells and fusing myeloma cells to form hybridomas. Alternatively, the selected polynucleotide is administered directly, such as by intramuscular injection, and expressed in vivo. The expressed protein generates a variety of protein-specific immune responses, including production of antibodies comparable to administration of the protein.

Preparations of polyclonal and monoclonal a antibodies specific for polypeptides encoded by a selected polynucleotide are made using standard methods known in the art. The antibodies specifically bind to epitopes present in the polypeptides encoded by polynucleotides disclosed in the Sequence Listing. Typically, at least 6, 8, 10, or 12 contiguous amino acids are required to form an epitope. Epitopes that involve non-contiguous amino acids may require a longer polypeptide, e.g., at least 15, 25, or 50 amino acids. Antibodies that specifically bind to human polypeptide encoded by the provided polypeptides should provide a detection signal at least 5-, 10-, or 20-fold higher than a detection signal provided with other proteins used in Western blots or other immunochemical assays. Preferably, antibodies that specifically bind polypeptides contemplated by the Prevention do not bind to other proteins immunochemical assays at detectable levels and can immunoprecipitate the specific polypeptide from solution.

The invention also contemplates naturally occurring antibodies specific for a polypeptide of the invention. For example, serum antibodies to ad polypeptide of the invention in a human population can be purified by methods well known in the art, e.g., by passing antiserum over a column to which the corresponding selected polypeptide or fusion protein is bound. The bound antibodies can then be eluted from the column, for example, using a buffer with a high salt concentration.

In addition to the antibodies discussed above, the invention also contemplates genetically engineered antibodies (e.g., chimeric antibodies, humanized antibodies, human antibodies produced by a transgenic (e.g., a transgenic mouse such as, the Xenomous™), antibody derivatives (e.g., single chain antibodies, antibody fragments (e.g., Fab, etc.)), according to methods well known in the art.

Polynucleotides or Arrays for Diagnostics

Polynucleotide arrays provide a high throughput technique that can assay a large number of polynucleotides in a sample. This technology can be used as a diagnostic and as tool to test for differential expression, e.g., to determined function protein. A variety of methods of producing arrays, as well as variations of these methods, are known in the art and contemplated for use in the invention. For example, arrays cane be created b spotting polynucleotide probes onto a substrate (e.g., glass, mitrocellulose, etc.) in a two-dimensional matrix or array having bound probes. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes, Double stranded polynucleotides, comprising the labeled sample polynucleotides bound to probe polynucleotides can be detected once the unbound portion of the sample is washed away. Alternatively, the polynucleotides of the test sample can be immobilized on the array, and the probes detectably labeled. Techniques for constructing arrays and methods of using these arrays are described in, for example, Schena et al. (19996) Proc. Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520, U.S. Pat. No. 5,599,695; EP 721 01-6; U.S. Pat. No. 5,556,752; WO 95/22058; and U.S. Pat. No. 5,631,734.

Arrays can be used to, for example, examine differential expression of genes and can be used to determine gene function. For example, arrays can be used to detect differential expression of a gene corresponding to a polynucleotide of the invention, where expression is compared between a test cell and control cell (e.g., cancer cells and normal cells). For example, high expression of a particular message in a cancer cell, which is not observed in a corresponding normal cell, can indicate a cancer specific gene product. Exemplary uses of arrays are further described in, for example, Pappalarado et al., Sem. Radiation Oncol. (1998) 8:217; and Ramsay Nature Biotechnol. (1998) 166:40. Furthermore, many variations on methods of detection using arrays are well within the skill in the art and within the scope of the present invention. For example, rather than immobilizing the probe to a solid support, the test sample can be immobilized on a solid support which is then contacted with the probe.

Differential Expression in Diagnosis

The polynucleotides of the invention can also be used to detect differences in expression levels between two cells, e.g., as a method to identify abnormal or diseased tissue in a human. For polynucleotides corresponding to profiles of protein families, the choice of tissue can be selected according to the putative biological function. In general, the expression of a gene corresponding to a specific polynucleotide is compared between a first tissue that is suspected of being diseased and a second, normal tissue of then human. The tissue suspected of being abnormal or diseased can be derived from a different tissue type of the human, but preferably it is derived from the same tissue type; for example, an intestinal polyp or other abnormal growth should be compared with normal intestinal tissue. The normal tissue can be the same tissue as that of the test sample, or any normal tissue of the patient, especially those that express the polynucleotide-related gene of interest (e.g., brain, thymus, testis, heart, prostate, placenta, spleen, small intestine, skeletal muscle, pancreas, and the mucosal lining of the colon). A difference between the polynucleotide-related gene, mRNA, or protein in the two tissues which are compared, for example, in molecular weight, amino acid or nucleotide sequence, or relative abundance, indicates a change in the gene, or a gene which regulates it, in the tissue of the human that was suspected of being diseased. Examples of detection of differential expression and its use in diagnosis of cancer are described in U.S. Pat. Nos. 5,688,641 and 5,677,125.

A genetic predisposition to disease in a hum an can also be detected by comparing expression levels of an mRNA or protein corresponding to a polynucleotide of the invention in a fetal tissue with levels associated in normal fetal tissue. Fetal tissues that are used for this purpose include but are not limited to, amniotic fluid, chorionic villi, blood and the blastomere of an in vitro-fertilized embryo. The comparable normal polynucleotide-related gene is obtained from any tissue. The mRNA or protein is obtained from a normal tissue of a human in which the polynucleotide-related gene is expressed. Differences such as alterations in the nucleotide sequence or size of the same product of the fetal polynucleotide-related gene or in RNA, or alterations in the molecular weight amino acid sequence, or relative abundance of fetal protein, can indicate a germline mutation in the polynucleotide-related gene of the fetus, which indicates a genetic predisposition to disease. In general, diagnostic, prognostic, and other methods of the invention based on differential expression involve detection of a level or amount of a gene product, particularly a differentially expressed gene product, in a test sample obtained from a patient suspected of having or being susceptible to a disease. (e.g. breast cancer, lung cancer, colon cancer and/or metastatic forms thereof), and comparing the detected levels to those levels found in normal cells (e.g., cells substantially unaffected by cancer) and/or other control cells (e.g., to differentiate a cancerous cell from a cell affected by dysplasia). Furthermore, the severity of the disease can be assessed by comparing the detected levels of a differentially expressed gene product with those levels detected in samples representing the levels of differentially expressed gene product associated width varying degrees of severity of disease it should be noted that use of the term “diagnostic” herein is not necessarily meant to exclude “prognostic” or “prognosis,” but rather is, used, as a matter of convenience.

The term “differentially expressed gene” is generally intended to encompass a polynucleotide that can, for example, include an open reading frame encoding a gene product (e.g., a polypeptide) and/or introns of such genes and adjacent 5′, and 3′ non-coding nucleotide sequences involved in the regulation of expression, up to about 20 kb beyond coding region, but possibly further in either direction. The gene an be introduced into an appropriate vector for extrachromosomal maintenance or for integration into a host genome. In general, a difference in expression level associated with a decrease in expression level of at least about 25%, usually at least about 50% to 75%, more usually at least about 90% or more is indicative of a differentially expressed gene of interest, i.e., a gene that is underexpressed or down-regulated in the test sample relative to a control sample. Furthermore, a difference impression level associated with an increase in expression of at least about 25%, usually at least about 50% to 75%, more usually at least that about 90% and a be at least about 1½-fold, usually at least about 2-fold to about 10-fold and can be about 100-fold to about 100-fold increase relative to a control sample is indicative of a differentially expressed gene of interest, i.e. an overexpressed or up-regulated gene.

“Differentially expressed polynucleotide” as used herein means a nucleic Acid molecule (RNA or DNA) comprising a sequence that represents a differentially expressed gene, e.g., the differentially expressed polynucleotide comprises a sequence (e.g., an open reading frame encoding a gene product that uniquely identifies a differentially expressed gene so that detection of the differentially expressed polynucleotide in a sample is correlated with the presence of a differentially expressed gene in a sample. “Differentially expressed polynucleotide” is also meant to encompass fragments of the disclosed polynucleotides, e.g., fragments retaining biological activity, as well as nucleic-acids to the disclosed polynucleotides.

Methods of the subject invention useful diagnosis or prognosis typically involve comparison of the abundance of a selected differentially expressed gene product in a sample of interest with that of a control to determine any relative differences in the expression of the gene product, where the difference can be measured qualitatively and/or quantitatively. Quantitation can be accomplished, for example, by comparing the level of expression produced detected in the sample with the amounts of product present in a standard curve. A comparison can be made visually; by using a technique such as densitometry, with or without computerized assistance; by preparing a representative library of cDNA clones of mRNA isolated from a test sample, sequencing the clones in the library to determine that number of cDNA clones corresponding to the same gene product, and analyzing the number of clones corresponding to that same gene product relative to the number of clones of the same gene product in a control sample; or by using an array to detect relative levels of hybridization to a selected sequence or set of sequences, and comparing the hybridization pattern to that of a control. The differences in expression are then correlated with the presence or absence of an abnormal expression pattern. A variety of different methods for determining the nucleic abundance in a sample are known to those of skill in the art (see, e.g., WO 97/27317).

In general, diagnostic assays of the invention involve detection of a gene product of a polynucleotide sequence (e.g., mRNA or polypeptide) that corresponds to a sequence of SEQ ID NOS:1-6010. The patient from whom the sample is obtained can be apparently healthy, susceptible to disease (e.g., as determined by family history or exposure to certain environmental factors), or can already be identified as having a condition in which altered expression of a gene product of the invention is implicated.

Diagnosis can be determined based on detected gene product expression levels of a gene product encoded by at least one preferably at least two or more, at least 3 or more, or at least 4 or more of the polynucleotides having a sequence set forth in SEQ ID NOS:1-6010, and can involve detection of expression of genies corresponding to all of SEQ ID NOS:1-6010 and/or additional sequences that can serve as additional diagnostic markers and/or reference sequences. Mere the diagnostic method is designed to detect the presence or susceptibility of a patient to cancer, the assay preferably involves detection of a gene product encoded by a gene corresponding to a polynucleotide that is differentially expressed in cancer. Examples of such differentially expressed polyucleotides are described in the Examples below. Given the provided polynucleotides and information regarding their relative expression levels provided herein, assays using such polynucleotides and detection of their expression levels in diagnosis and prognosis will be readily apparent to the ordinarily skilled artisan.

Any of a variety of detectable labels can be used in connection with the various embodiments of the diagnostic methods of the invention. Suitable detectable labels include fluorochromes (e.g., fluorescein isothiocyanate (FITC) rhodamine, Texas Red, phycoeythrin, allophycocyanin, 6-carboxyfluorescein (6-FAM), 2′,7-diethoxy-4′,5′-dichloro-6-carboxyfluorescein, 6-carboxy-X-rhodamine (ROX), 6-carboxy-2′,4′,7′, 4,7-hexachlorofluorescein (HEX), 5-carboxyfluorescein (5-FAM) or N,N,N′,N′-tetramethyl-6-carboxyrhodamine (TAMRA)), radioactive labels, (e.g. 32P, 35S, 3H, etc.), and the like. The detectable label can involve a two stage systems (e.g., biotin-avidin, hapten-anti-hapten antibody, etc.).

Reagents specific for the polynucleotides and polypeptides of the invention, such as antibodies and nucleotide probes, can be supplied in a kit for detecting the presence of an expression product in, a biological sample. The kit can also contain buffers or labeling components, as well as instructions for using the reagents to detect and quantify expression products in the biological sample. Exemplary embodiments of the diagnostic methods of the invention are described below in more detail.

Polypeptide detection in diagnosis. In one embodiment, the test sample is assayed for the level of a differentially expressed polypeptide. Diagnosis can be accomplished using any of a number of methods to determine the absence or presence or altered amounts of the differentially expressed polypeptide in the test sample. For example, detection can utilize staining of cells or histological sections with labeled antibodies, performed in accordance with conventional methods. Cells can be permeabilized to stain cytoplasmic molecules. In general, antibodies that specifically bind a differentially expressed polyeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods of qualitative or quantitative detection of levels or amounts of differentially expressed polypeptide can be used, for example, ELISA, western blot, immunoprecipitation, radioimmunoassay, etc.

mRNA detection. The diagnostic methods of the invention can also or alternatively involve detection of mRNA encoded by a gene corresponding to a differentially expressed polynucleotide of the invention. Any suitable qualitative or quantitative methods known in the art for detecting specific mRNAs can be used. mRNA can be detected by, or example, in situ hybridization in tissue sections, by reverse transcriptase-PCR, or in Northern blots containing poly A+ mRNA. One of skill in the art can readily use these methods to determine differences in the size or amount of mRNA transcripts between two samples. mRNA expression levels in a sample can also be determined by generation of a library of expressed sequence tags (ESTs) from the sample where the EST library is representative of sequences present in the sample (Adams, et al., (1991) Science 252:1651). Enumeration of the relative representation of ESTs within the library can be used to approximate the relative representation of the gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein. Alternatively, gene expression in a test sample can be performed using serial analysis of gene expression (SAGE) methodology (e.g., Velculescu et al., Science (1995) 270:484) or differential display (DD) methodology (see, e.g., U.S. Pat. No. 5,776,683 and U.S. Pat. No. 5,807,680).

Alternatively, gene expression can be analyzed using hybridization analysis. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Use of a single gene in diagnostic applications. The diagnostic methods of the invention can focus on the expression of a single differentially expressed gene. For example, the diagnostic method can involve a differentially expressed gene or a polymorphism of such a gene (e.g., a polymorphism in a coding region or control region) that is associated with disease. Disease-associated polymorphisms can include deletion or truncation of the gene, mutations that alter expression level and/or affect activity of the encoded protein, etc.

A number of methods are available for an analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express a differentially expressed gene can be used as a source of mRNA, which can be assayed directly or reverse transcribed into cDNA for analysis.

The nucleic acid can be amplified by conventional techniques, such as the polymerase chain reaction (PCR), to provide sufficient amounts for analysis, and a detectable label can be included in the amplification reaction (e.g., using a detectably labeled primer or detectably labeled oligonucleotides) to facilitate detection. Alternatively, various methods are also known in the art that utilize oligonucleotide ligation as a means of detecting polymorphisms, see, e.g., Riley et al., Nucl. Acids Res. (1990) 18:2887; and Delahunty et al., Am. J. Hum. Genet. (1996) 58:1239.

The amplified or cloned sample nucleic acid can be analyzed by one of a number of methods known in the art. The nucleic acid can be sequenced by dideoxy or other methods, and the sequence of bases compared to a selected sequence, e.g., to a wild-type sequence. Hybridization with the polymorphic or variant sequence can also be used to determine its presence in a sample (e.g., by Southern blot, dot blot, etc.). The hybridization pattern of a polymorphic or variant sequence and a control sequence to an array of oligonucleotide probes immobilized on a solid support, as described in U.S. Pat. No. 5,445,934, or in WO 95/35505, can also be used as a means of identifying polymorphic or variant sequences associated with disease. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Alternatively, where a polymorphism creates or destroys a recognition site for a restriction endonuclease, the sample is digested with that endonuclease, and the products size fractionated to determine whether the fragment was digested. Fractionation is performed by gel or capillary electrophoresis, particularly acrylamide or agarose gels.

Screening for mutations in a gene can be based on the functional or antigenic characteristics of the protein. Protein truncation assays are useful in detecting deletions that can affect the biological activity of the protein. Various immunoassays desired to detect polymorphisms in proteins can be used in screening. Where many diverse genetic mutations lead to a particular disease phenotype, functional protein assays have proven to be effective screening tools. The activity of the encoded protein can be determined by comparison with the wildtype protein.

Diagnosis, Prognosis, Assessment of Therapy (Therametrics), and Management of Cancer

The polynucleotides of the invention, as well as their gene products, are of particular interest as genetic or biochemical markers (e.g., in blood or tissues) that will detect the earliest changes along the carcinogenesis pathway and/or to monitor the efficacy of various therapies and preventive interventions. For example, the level of expression of certain polynucleotides can be indicative of a poorer prognosis, and therefore warrant more aggressive chemo- or radio-therapy for a patient or vice versa. The correlation of novel surrogate tumor specific features with response to treatment and outcome in patients can define prognostic indicators that allow the design of tailored therapy based on the molecular profile of the tumor. These therapies include antibody targeting, antagonists (e.g., small molecules), and gene therapy. Determining expression polynucleotides and comparison of a patient's profile with known expression in normal tissue and variants of the disease allows a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the genes corresponding to the polynucleotides of the invention are staging of the cancerous disorder, and grading the nature of the cancerous tissue.

The polynucleotides that correspond to differentially expressed genes, as well as their encoded gene products, can be useful to monitor patients having or susceptible to cancer to detect potentially malignant events at a molecular level before they are detectable at a gross morphological level. In addition, the polynucleotides of the invention, as well as the genes corresponding to such polynucleotides, can be useful as therametrics, e.g., to assess the effectiveness of therapy by using the polynucleotides or their encoded gene products, to assess, for example, tumor burden in the patient before during, and after therapy.

Furthermore, a polynucleotide identified as corresponding to a gene that is differentially expressed in, and thus is important for one type of cancer can also have implications for development or risk of development of other types of cancer, e.g., where a polynucleotide represents a gene differentially expressed across various cancer types. Thus, for example, expression of a polynucleotide corresponding to a gene that has clinical implications for metastatic colon cancer can also have clinical implications for stomach cancer or endometrial cancer.

Staging. Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

The polynucleotide of the invention can facilitate fine-tuning of the staging process by identifying markers for the aggresivity of a cancer, e.g., the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

Grading of cancers. Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors being more aggressive than well-differentiated or low-grade tumors. The following guidelines are generally used for grading tumors. 1) GX Grade cannot be assessed; 2) G1 Well differentiated; 3) G2 Moderately well differentiated; 4) G3 Poorly differentiated; 5) G4 Undifferentiated. The polynucleotides of the invention can be especially valuable in determining the grade of the tumor, as they not only can aid in determining the differentiation status of the cells of a tumor, they can also identify factors other than differentiation that are valuable in determining the aggressiveness of a tumor, such as metastatic potential.

Detection of colon cancer. The polynucleotides corresponding to genes that exhibit the appropriate expression pattern can be used to detect colon cancer in a subject. Colorectal cancer is one of the most common neoplasms in humans and perhaps the most frequent form of hereditary neoplasia. Prevention and early detection are key factors in controlling and curing colorectal cancer. Colorectal cancer begins as polyps which are small, benign growths of cells that form on the inner lining of the colon. Over a period of several years, some of these polyps accumulate additional mutations and become cancerous. Multiple familial colorectal cancer disorders have been identified, which are summarized as follows 1) Familial adenomatous polyposis (FAP); 2) Gardner's syndrome, 3) Hereditary nonpolyposis colon cancer (HNPCC); and 4) Familial colorectal cancer in Ashkenazi Jews. The expression of appropriate polynucleotides of the invention can be used in the diagnosis, prognosis and management of colorectal cancer Detection of colon cancer can be determined using expression levels of any of these sequences alone or in combination with the levels of expression. Determination of the aggressive nature and/or the metastatic potential of a colon cancer can be determined by comparing levels of one or more polynucleotides of the invention and comparing total levels of another sequence known to vary in cancerous tissue, e.g., expression of p53, DCC ras, lor FAP (see, e.g., Fearon E R, et al., Cell (1990) 61(5):759; Hamilton S R et al., Cancer (1993) 72:957; Bodmer W, et al., Nat Genet (1994) 4(3):217; Fearon E R, Ann N Y Acad Sci. (1995) 768:101). For example, development of colon cancer can be detected by examining the ratio of any of the polynucleotides of the invention to the levels of oncogenes (e.g., ras) or tumor suppressor genes (e.g., PAP or p53). Thus, expression of specific marker polynucleotides can be use d to discriminate between normal and cancerous colon tissue, to discriminate between colon cancers with different cells of origin, to discriminate between colon cancers with different potential metastatic rates, etc. For a review of markers of cancer, see, e.g., Hanahan et al. (2000) Cell 100:57-70.

Detection of prostate cancer. The polynucleotides and their corresponding genes and gene products exhibiting the appropriate differential expression pattern can be used to detect prostate cancer in a subject. Over 95% of primary prostate cancers are adenocarcinomas. Signs and symptoms may include: frequent urination, especially at night; inability to urinate; trouble starting or holding back urination; a weak or interrupted urine flow; and frequent pain or stiffness in the lower back, hips or upper thighs.

Many of the signs and symptoms of prostate cancer can be caused by a variety of other non-cancerous conditions. For example, one common cause of many of these signs and symptoms is a condition called benign prostatic hypertrophy, or BPH. In BPH, the prostate gets bigger and may block the flow of urine or interfere with sexual function. The methods and compositions of the invention can be used to distinguish between prostate cancer and such non-cancerous conditions. The methods of the invention can be used in conjunction with conventional methods of diagnosis, e.g., digital rectal exam and/or detection of the level of prostate specific antigen (PSA), a substance produced and secreted by the prostate.

Detection of breast cancer. The majority of breast cancers are adenocarcinoma subtypes, which can be summarized as follows: 1) ductal carcinoma in situ (DCIS), including comedocarcinoma, 2) infiltrating (or invasive) ductal carcinoma (IDC); 3) lobuar carcinoma in situ (LCIS); 4) infiltrating (or invasive) lobular carcinoma (ILC); 5) inflammatory breast cancer; 6) medullary carcinoma; 7) mucinous carcinoma; 8) Paget's disease of the nipple 9) Phyllodes tumor; and 10) tubular carcinoma;

The expression of polynucleotides of the invention can be used in the diagnosis and management of breast cancer, as well as to distinguish between types of breast cancer. Detection of breast cancer can be determined using expression levels of any of the appropriate polynucleotides of the invention, either alone or in combination. Determination of the aggressive nature and/or the metastatic potential of a breast cancer can also be determined by comparing levels of one or more polynucleotides of the invention and comparing levels of another sequence known to vary in cancerous tissue, e.g., ER expression. In addition, development of breast cancer can be detected by examining the ratio of expression of a differentially expressed polynucleotide to the levels of steroid hormones (e.g., testosterone or estrogen) or to other hormones (e.g., growth hormone, insulin). Thus, expression of specific marker polynucleotides can be used to discriminate between normal and cancerous breast tissue, to discriminate between breast cancers with different cells of origin, to discriminate between breast cancers with different potential metastatic rates, etc.

Detection of lung cancer. The polynucleotides of the invention can be used to detect lung cancer in a subject. Although there are more than a dozen different cancer, the two main types of lung cancer are small cell and nonsmall cell, which encompass about 90% of all lung cancer cases. Small cell carcinoma (also called oat cell carcinoma) usually starts in one of the larger bronchial tubes, grows fairly rapidly, and is likely to be large by the time of diagnosis. Nonsmall cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large. Adenocarcinoma starts growing near the outside surface of the lung and can vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

The polynucleotides of the invention, e.g., polynucleotides differentially expressed in normal cells ver cancerous lung cells (e.g., tumor cells of high or low metastatic potential) or between types of cancerous lung cells (e.g., high metastatic versus low metastatic), can be used to distinguish types of lung cancer as well as its specific to a certain patient's cancer and selecting an appropriate therapy. For example, if the patient's biopsy expresses a polynucleotide that is associated with a low metastatic potential, it may justify leaving a larger portion of the patient's lung in surgery to remove the lesion. Alternatively, a smaller lesion with expression of a polynucleotide that is associated with high metastatic potential may justify a more radical removal of lung tissue and/or the surrounding lymph nodes, even if no metastasis can be identified through pathological examination.

Use of Polynucleotides to Screen for Peptide Analog and Antagonists

Polypeptides encoded by the instant polynucleotides and corresponding full-length genes can be used to screen peptide libraries to identify binding partners, such as receptors, from among the encoded polypeptides. Peptide libraries can be synthesized according to methods known in the art (see, e.g., U.S. Pat. No. 5,010,175, and WO 91/17823). Agonists or antagonists of the polypeptides of the invention can be screened using any available method known in the art, such as signal transduction, antibody binding receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of biding irreversibly to the polypeptide can be added in concentrations on the order of the native concentration.

Such screening and experimentation can lead to identification of a novel polypeptide binding partner, such as a receptor, encoded by a gene or a cDNA corresponding to a polynucleotide of the invention and at least one peptide agonist or antagonist of the novel binding partner. Such agonists and antagonists can be used to modulate, enhance, or inhibit receptor function in cells to which the receptor is native, or in cells possess the receptor as a result of genetic engineering. Further, if the novel receptor shares biologically important characterstics with a known receptor, information about agonist/antagonist binding can facilitate development of improved agonists/antagonists of the known receptor.

Pharmaceutical Compositions and Uses

Pharmaceutical compositions can comprise polypeptides, receptors that specifically bind a polypeptide produced by a differentially expressed gene (e.g., antibodies, or polynucleotides (including antisense nucleotides and ribozymes) of the claimed invention in a therapeutically effective amount. The compositions can be used to treat primary tumors as well as metastases of primary tumors. In addition the pharmaceutical compositions can be used in conjunction with conventional methods of cancer treatment, e.g., to sensitize tumors to radiation or conventional chemotherapy.

Where the pharmaceutical composition comprises a receptor (such as an antibody) that specifically binds to a gene product encoded by a differentially expressed gene, the receptor can be coupled to a drug for delivery to a treatment site or coupled to a detectable label to facilitate imaging of a site comprising colon cancer cells. Methods for coupling antibodies to drugs and detectable labels are well known in the art, as are methods for imaging using detectable labels.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgement of the clinician. For purposes of the present invention, an effective dose will generally be from about 0.01 mg/kg to 50 mg/g or 0.05 mg, 1 g to about 10 mg/kg of the DNA, constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, poly lactic acid, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are pared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Delivery Methods

Once formulated, the compositions of the invention can be (1) administered directly to the subject (e.g., as polynucleotide or polypeptides); or (2) delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, intratumorally or to the interstitial space of a tissue. Other modes of administration include oral and pulmonary administration suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in, e.g., WO 93/14778. Examples of cells useful in ex vivo applications include for example stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation polybrene mediated transfection, protoplast fusion, electroporation, encapsulation of the polynucleotide(s) in liposomes, and direct microinjection of the DNA into nuclei, all well known in the art.

Once differential expression of a gene corresponding to a polynucleotide of the invention has been found to correlate with a proliferative disorder, such as neoplasia, dysplasia, and hyperplasia, the disorder can be amenable to treatment by administration of a therapeutic agent based on the provided polynucleotide, corresponding polypeptide or other corresponding molecule (e.g., antisense, ribozyme, etc.). In other embodiments, the disorder can be amenable to treatment by administration of a small molecule drug that, for example, serves as an inhibitor (antagonist) of the function of the encoded gene product of a gene having increased expression in cancerous cells relative to normal cells or as an agonist for gene products that are decreased in expression in cancerous cells (e.g., to promote the activity of gene products that act as tumor suppressors).

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic composition agents of the invention includes local or systemic administration, including injection, oral administration, particle gun or catheterized administrator, and topical administration. Preferably, the therapeutic polynucleotide composition contains an expression construct comprising a promoter operably linked to a polynucleotide of at least 12, 22, 25, 30, or 35 contiguous nt of the polynucleotide of the invention. Various methods can be used to administer the therapeutic composition directly to a specific site in the body. For example, a small metastatic lesion is located and the therapeutic composition injected several times in several different locations within the body of tumor. Alternatively, arteries that serve a tumor are identified, and the therapeutic composition injected into such an artery in order o deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods.

Targeted delivery of therapeutic compositions containing antisense polynucleotide, subgenomic polynucleotides or antibodies to specific tissues can also be used. Receptor-mediated DNA delivery techniques are described in, for example, Findeis et al., Trends Biotechnol. (993) 11:202; Chiou et al., Gene Therapeutics: Methods And Applications Of Direct Gene Transfer (J. A. Wolff ed.) (1994); Wu et al., J. Biol. Chem. (1988) 263:621; Wu et al., J. Biol. Chem. (1994) 269:542, Zenke et al., Proc. Natl. Acad. Sci. (USA)(1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 micrograms to about 2 mg, about 5 micrograms to about 500 micrograms, and about 20 micrograms to about 1 micrograms of DNA can also be used during a gene therapy protocol Factors such as method of action (e.g., for enhancing or inhibiting levels of the encoded gene product) and efficacy of transformation and expression are considerations which will affect the dosage required for ultimate efficacy of the antisense subgenomic polynucleotides. Where greater expression is desired over a larger area of tissue, larger amounts of antisense subgenomic polynucleotides or the same amounts readministered in a successive protocol of administrations, or several administrations to different adjacent or close tissue portions of, for example a tumor site, may be required to effect a positive therapeutic outcome. In all cases, routine experimentation in clinical trials will determine specific ranges for optimal therapeutic effect. For polynucleotide related genes encoding polypeptides or proteins with antiinflammatory activity, suitable use, doses, and administration are described in U.S. Pat. No. 5,654,173.

The therapeutic polynucleotides and polypeptides of the present invention can be delivered using gene delivery vehicles. The gene delivery vehicle can be of viral or non-viral origin (see generally Jolly, Cancer Gene Therapy (1994) 1:51; Kimura, Human Gene Therapy (1994) 5:845; Connelly, Human Gene Therapy (1995) 1:185; and Kaplitt Nature Genetics (1994) 6:148) Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698, WO 93/25234; U.S. Pat. No. 5,219,740, WO 93/11230, WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCCVR-1246) and Venezuelan equine encephalitis virus (ATCC VR-923; ATCC VR-1250; ATCC VR 1249, ATCC VR-532), and adeno-associated virus (AAV) vectors (see; e.g., WO 94/12649, WO 93/03769; WO 93/19191; WO 94/28938; WO 95/11984 and WO 95/00655). Administration of DNA linked to killed adenovirus, as described in Curiel, Hum. Gene Ther. (1992) 3:147, can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see e.g., Wu, J. Biol. Chem. (1989) 264:16985); eukaryotic cell delivery vehicles cells (see, e.g., U.S. Pat. No. 5,814,482; WO 95/07994; WO 96/17072; WO 95/30763, and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell. Biol. (1994) 14:2411, and in Woffendin, Proc. Natl. Acad. Sci. (1994) 91:1581.

Further non-viral delivery suitable for use includes mechanical delivery systems such as the approach described in Woffendin et al., Proc. Natl. Acad. Sci. USA (1994) 91(24):11581. Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

The present invention will now be illustrated by reference to the following examples which set forth particularly advantageous embodiments. However, it should be noted that these embodiments are illustrative and are not to be construed as restricting the invention in any way.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. It will be readily apparent to those skilled in the art that the formulations dosages methods of administration, and other parameters of this invention may be further modified or substituted in various ways without departing from the spirit and scope of the invention. Efforts have been made to ensure accuracy with respect to numbers used (e.g., amounts, temperature, etc.) but some experimental errors and deviations should be accounted for Unless indicated otherwise, parts are parts by weight, molecular weight is weight average molecular weight, temperature is in degrees Centigrade, and pressure is at or near atmospheric.

### Example 1

### Source of Biological Materials and Overview of Novel Polynucleotides Expressed by the Biological Materials

Candidate polynucleotides were obtained from cDNA libraries generated from selected cell lines and patient tissues. In order to obtain the candidate polyucleotides, mRNA was isolated from several selected cell lines and patient tissues, and used to construct cDNA libraries. The cells and issues that served as sources for these cDNA libraries are summarized in Table 1 below.

Human colon cancer cell line Km12L4-A (Morikawa, et al., Cancer Research (1988) 48:6863) is derived from the KM12C cell line. The KM12 C cell line (Morikawa et al. Cancer Res. (1988) 48:1943-1948), which is poorly metastatic (low metastatic) was established in culture from a Dukes' stage B2 surgical specimen (Morikawa et al. Cancer Res. (1988) 48:6863). The KM12L4-A is a highly metastatic subline derived from KM12C (Yeatman et al. Nucl. Acids. Res. (1995) 23:4007; Bao-Ling et al. Proc Annu. Meet Am. Assoc. Cancer. Res. (1995) 21:3269). The KM12C and KM12C-derived cell lines (e.g., KM12L4, KM12L4-A, etc.) are well-recognized in the art as a model cell line for the study of colon cancer (see, e.g. Moriakawa et al., supra; Radinsky et al. Clin. Cancer Res. (1995) 1:19; Yeatman et al., (1995) supra; Yeatman et al. Clin. Exp. Metastasis (1996) 14:246).

The MDA-MB-231 cell line (Brinkley et at. Cancer Res. (1980) 40:3118-3'129) was originally isolated from pleural effusions (Cailleau, J. Natl. Cancer. Inst. (1974) 53:661), is of high metastatic potential, and forms poorly differentiated adenocarcinoma grade II in nude mice consistent with breast carcinoma. The MCF7 cell line was derived from a pleural effusion of a breast adenocarcinoma and is non-metastatic. The MV-522 cell line is derived from a human lung carcinoma and is of high metastatic potential. The UCP3 cell line is a low metastatic human lung carcinoma cell line; the MV-522 is a high metastatic variant of UCP-3. These cell lines are well-recognized in the art as models for the study of human breast and lung cancer (see, e.g., Chandrasekaran et al., Cancer Res. (1979) 39:870 (MDA MB 231 and MCF-7); Gastpar et al. J Med Chem (1,998) 41:4965, (MDA-MB-231 and MCF-7); Ranson et al., Br J Cancer (1998) 77:1586 (MDA-MB-231 and MCF-7); Kuang et al., Nucleic Acids Res. (1998) 26:1116 (MDA-MB-231 and MCF-7); Varki et al., Int J Cancer (1987) 40:46 (UCP-3); Varki et al., Tumour Biol. (1990) 11:327; (MV-522 and UCP-3); Varki et at, Anticancer Res. (1990) 10:637; (MV-522); Kelner et al., Anticancer Res (1995) 15:867 (MV-522); and Zhang et al., Anticancer Drugs (11997) 8-696 (MV522)).

The samples of libraries 15-20 are derived from two different patients (UC#2, and UC#3). The bFGF-treated HMVEC, were prepared by incubation with bFGF at 10 ng/ml for 2 hrs the VEGF-treated HMVEC were prepared by incubation with 20 ng/ml VEGF for 2 hrs. Following incubation with the respective growth factor, the cells were washed and, lysis buffer added for RNA preparation. The GRRpz and WOca cell lines were provided by Dr. Donna M. Peehl, Department of Medicine, Stanford University School of Medicine. GRRpz was derived from normal prostate epithelium. The WOca cell line is a Gleason Grade 4 cell line.

TABLE 1
Description of cDNA Libraries

| | | Number of |
|---|---|---|
| Library | | Clones in |
| (lib #) | Description | Library |
| 0 | Artificial library composed of deselected clones (clones with no | 673 |
| | associated variant or cluster) | |
| 1 | Human Colon Cell Line Km12 L4: High Metastatic Potential (derived | 308731 |
| | from Km12C) | |
| 2 | Human Colon Cell Line Km12C: Low Metastatic Potential | 284771 |
| 3 | Human Breast Cancer Cell Line MDA-MB-231: High Metastatic | 326937 |
| | Potential; micro-mets in lung | |
| 4 | Human Breast Cancer Cell Line MCF7: Non Metastatic | 318979 |
| 8 | Human Lung Cancer Cell Line MV-522: High Metastatic Potential | 223620 |
| 9 | Human Lung Cancer Cell Line UCP-3: Low Metastatic Potential | 312503 |
| 12 | Human microvascular endothelial cells (HMEC) - UNTREATED | 41938 |
| | (PCR (OligodT) cDNA library) | |
| 13 | Human microvascular endothelial cells (HMEC) - bFGF TREATED | 42100 |
| | (PCR (OligodT) cDNA library) | |
| 14 | Human microvascular endothelial cells (HMEC) - VEGF TREATED | 42825 |
| | (PCR (OligodT) cDNA library) | |
| 15 | Normal Colon - UC#2 Patient (MICRODISSECTED PCR (OligodT) | 282722 |
| | cDNA library) | |
| 16 | Colon Tumor - UC#2 Patient (MICRODISSECTED PCR (OligodT) | 298831 |
| | cDNA library) | |
| 17 | Liver Metastasis from Colon Tumor of UC#2 Patient | 303467 |
| | (MICRODISSECTED PCR (OligodT) cDNA library) | |
| 18 | Normal Colon - UC#3 Patient (MICRODISSECTED PCR (OligodT) | 36216 |
| | cDNA library) | |
| 19 | Colon Tumor - UC#3 Patient (MICRODISSECTED PCR (OligodT) | 41388 |
| | cDNA library) | |
| 20 | Liver Metastasis from Colon Tumor of UC#3 Patient | 30956 |
| | (MICRODISSECTED PCR (OligodT) cDNA library) | |
| 11 | GRRpz Cells derived from normal prostate epithelium | 164801 |
| 22 | WOca Cells derived from Gleason Grade 4 prostate cancer epithelium | 162088 |
| 23 | Normal Lung Epithelium of Patient #1006 (MICRODISSECTED PCR | 306198 |
| | (OligodT) cDNA library) | |
| 24 | Primary tumor, Large Cell Carcinoma of Patient #1006 | 309349 |
| | (MICRODISSECTED PCR (OligodT) cDNA library) | |
| 25 | Normal Prostate Epithelium from Patient IF97-26811 | 279444 |
| 26 | Prostate Cancer Epithelium Gleason 3 + 3 Patient IF97-26811 | 269406 |
| 27 | formal Breast Epithelium from Patient 515 | 239494 |
| 28 | Primary Breast tumor from Patient 515 | 259960 |
| 29 | Lymph node metastasis from Patient 515 | 326786 |
| 30 | formal Prostate Epithelium from Chiron Patient ID 884 | 298431 |
| 31 | Prostate Cancer Epithelium (Gleason 4 + 4) from Chiron Patient ID 884 | 331941 |

Characterization of Sequences in the Libraries

After using the software program Phred (ver 0.500925.c, Green and Weing @ 1993-2000) to select polynucleotides having the best quality sequence, the polynucleotides were compared against the public databases to identity any homolgous sequences. The sequences of the isolated polynucleotides were first masked to eliminate low complexity sequences using the BLASTX masking program (Claverie “Effective Large-Scale Sequence Similarity Searches,” In: Computer Methods for Macromolecular Sequence Analysis, Doolittle, ed., Meth. Enzymol. 266:212-227 Academic-Press, NY, NY (1996); see particularly Claveric in “Automated DNA Sequencing and Analysis Techniques” Adams et al., eds., Chap. 36, p 267Academic Press, San Diego, 1994 and Clavene et al Comput. Chem. (1993) 17:191). Generally, masking does not influence the final search results, except to eliminate sequences of relatively little interest due to their low complexity, and to eliminate multiple “hits” based on similarity to repetitive regions common to multiple sequences, e.g., Alu repeats. The remaining sequences were then used in a BLASTN vs. GenBank search; sequences that exhibited greater than 70% overlap; 99% identity, and a p value of less than 1×10e-40 were discarded. Sequences from this search also were discarded if the inclusive parameters were met, but the sequence was ribosomal or vector-derived.

The resulting sequences from the previous search were classified into three groups (1, 2 and 3 below) and searched in a BLASTX vs. NRP (non-redundant proteins) database search: (1) unknown (no hits in the GenBank search), (2) weak similarity (greater than 45,% identity and p value of less than 1×10e-5), and (3) high similarity; (greater than 60% overlap, greater than 80% identity and p value less than 1×10e-5). Sequences having greater than 70°% overlap, greater than 99% identity, and p value of less than 1×10e-40 were discarded.

The remaining sequences were classified as unknown (no hits), weak similarity, and high n similarity (parameters as above). Two searches were performed on these sequences. First, a BLAST vs. EST database search was performed and sequences with greater than 99% overlap greater than 99% similarity and a p value of less than 1×10e-40 were discarded. Sequences with a p value of less than 1×10e-65 when compared to a database sequence of human origin were also excluded. Second, a BLASTN vs. Patent GeneSeq database was performed and sequences having greater than 99% identity p value less than 1×10e-40, and greater than 99% overlap were discarded.

The remaining sequences were subjected to screening using other rules and redundancies in the dataset sequences with a p value of less than c 1×10e-1 in relation to 9a database sequence of human origin were specifically excluded. The final result provided the 8064 sequences listed as SEQ ID NOS:1-6010 in the accompanying Sequence Listing and summarized in Table 2 (inserted prior to claims). Each identified polynucleotide represents sequence from at least a partial mRNA transcript.

### SUMMARY OF POLYNUCLEOTIDES OF THE INVENTION

Table 2 (inserted prior to claims) provides a summary of polynucleotides isolated as described. Specifically, Table 2 provides) the SEQ ID NO (“SEQ ID”) assigned to each sequence for use in the present specification; 2) the Cluster Identification No. (“CLUSTER”); 3) the Sequence Name assigned to each sequence; 3) the sequence name (“SEQ NAME”) used as an internal identifier of the sequence; 4) the orientation of the sequence (“ORIENT”) (either forward (F) or reverse (R)); 5) the name assigned to the Clone from which the sequence was isolated (“CLONE ID”), and the name of the library from which the sequence was isolated (“LIBRARY”), where the notation indicates that name of the cell line or patient sample (e.g., UC2-NormColon indicates the sequence was isolated from normal colon tissue of the patient assigned the identification UC#2). Because at least some of the provided polynucleotides represent partial mRNA transcripts, two or more polynucleotides may represent different regions of the same mRNA transcript and the same gene and/or may be contained within the same clone. Thus, for example, if two or more SEQ ID NOS: are identified as belonging to the same clone, then either sequence can be used to obtain the full-length mRNA or gene

### Example 2

### Results of Public Database Search to Identify Function of Gene Products

SEQ ID NOS:1-6010 were translated in all three reading frames, and the nucleotide sequences and translated amino acid sequences used as query sequences to search for homologous sequences in either the GenBank (nucleotide sequences) or Non-Redundant Protein (amino acid sequences) databases. Query and individual sequences were aligned using the BLAST 2.0 programs, available over the world wide at a site sponsored by the National Center for Biotechnology Information, which is supported by the National Library of Medicine and the National Institutes of Health (see also Altschul, et al. Nucleic Acids Res. (1997) 25:33389-3402). The sequences were masked to various extents to prevent searching of repetitive sequences or poly-A sequences, using the BLASTX program for masking low complexity as described above in Example 1.

Tables 3A and 3B (inserted prior to claims) provides the alignment summaries having a p value 1×10e-2 or less indicating substantial homology between the sequences of the present invention and those of the indicated public databases. Specifically, Table 3A provides the SEQ ID NO of the query sequence, the accession number of the GenBank database entry of the homologous sequence, and the individual p value of each alignment. Table 3A provides the SEQ ID NO of the query sequence, the accession number of the Non-Redundant Protein database entry of the homologous sequence, and the individual p value of each alignment. The alignments provided in Tables 3A and 3B are the best available aliment to a DNA or amino acid sequence at a time just prior to filing of the present specification. The activity of the polypeptide encoded by the SEQ ID NOS listed in these tables can be extrapolated to be substantially the same or substantially similar to the activity of the reported nearest neighbor or closely related sequence. The accession number of the nearest neighbor is reported, providing a publicly available reference to the activities and functions exhibited by the nearest neighbor. The public information regarding the activities and functions of each of the nearest neighbor sequences is incorporated by reference in this application. Also incorporated by reference is all publicly available information regarding the sequence, as well as the putative and actual activities and functions of the nearest neighbor sequences listed in Tables 3A and 3B and their related sequences. The search program and database used for the alignment, as well as the calculation of the p value are also indicated.

Full length sequences or fragments of the polynucleotide sequences of the nearest neighbors can be used as probes and primers to identify and isolate the full length sequence of the corresponding polynucleotide. The nearest neighbors can indicate a tissue or cell type to be used to construct a library for the full-length sequences of the corresponding polynucleotides.

### Example 3

### Members of Protein Families

SEQ ID NOS:1-6010 were used to conduct a profile search as described in the specification above. Several of the polynucleotides of the invention were found to encode polypeptides having characteristics of a polypeptide belonging to a known protein family (and thus represent members of these protein families) and/or comprising a known functional domain Table 4 (inserted before claims), provides the SEQ ID NO: of the query sequence, the Sequence Name, the Cluster to which the sequence is assigned, a brief description of the profile hit, the orientation (Direction, “Dir”) of the query sequence with respect to the individual sequence) where forward (for) indicates that the alignment is in the same direction (left to right) as the sequence provided in the Sequence Listing and reverse (rev) indicates that the alignment is with a sequence complementary to the sequence provided in the Sequence Listing) and the score of the profile hit.

Some polynucleotides exhibited multiple profile hits where the query sequence contains overlapping profile regions, and/or where the sequence contains two different functional domains. Each of the profile hits of Table 4 is described in more detail below. The acronyms for the profiles (provided in parentheses) are those used to identify the profile in the Pfam, Prosite, and InterPro databases. The Pfam database can be accessed through web sites supported by Genome Sequencing Center at the Washington University School of Medicine or by the European Molecular Biology Laboratories in Heidelberg, Germany. The Prosite database can be accessed at the ExPASy Molecular Biology Server on the internet. The InterPro database can be accessed at a web site supported by the EMBL European Bioinformatics Institute. The public information available on the Pfam, Prosite, and InterPro databases regarding the various profiles, including but not limited to the activities, function, and consensus sequences of various proteins families and protein domains, is incorporated herein by reference.

Seven Transmembrane Integral Membrane Proteins—Rhodopsin Family (7tm_{—}1; Pfam Accession No. PF00001) SEQ ID NOS:2973, 5467, and 5508 correspond to sequences encoding a polypeptides that are members of the seven transmembrane (7tm) receptor rhodopsin family. G-protein coupled receptors of the (7tm) rhodopsin family (also called R7G) are an extensive group of hormones, neurotransmitters, and light receptors which transduce extacellular signals by interaction with guanine nucleotide-binding (G) proteins (Strosberg, Eur. J. Biochem. (1991) 196:1; Kerlavage, Curr. Opin. Struct. Biol. (1991) 1:394; Probst et al., DNA Cell Biol. (1992) 11:1; Savarese et al., Biochem J. (1992) 283:1. The consensus pattern that contains the conserved triplet and that also spans the major part of the third transmembrane helix is used to detect this widespread family of proteins; [GSTALIVMFYWC]-[GSTANCPDE]-{EDPKRH}-x(2)-[LIVMNQGA]-x(2)-[LIVMFT]-[GSTANC]-[LIVMFYWSTAC]-[DENH]-R-[FYWCSH]-x(2)-[LIVM].

Ank Repeats(ANK; Pfam Accession No. PF0023). SEQ ID NOS:445, 487 and 3013 represent polynucleotides encoding Ark repeat-containing proteins. The ankyrin motif is a 33 amino acid sequence named after the protein ankyrin which has 24 tandem 33-amino-acid motifs. Ank repeats were originally identified in the cell-cycle-control protein cdc10 (Breeden et al., Nature (1987) 329:651). Proteins containing ankyrin repeats include ankyrin, myotropin, I-kappaB proteins, cell cycle protein cdc10, the Notch receptor (Matsuno et al., Development (1997) 124(21):4265); G9a (or BAT8) of the class III region of the major histocompatibility complex (Biochem J. (1993) 290:811-818); FABP, GABP, 53BP2, Lin12, glp-1, SW14, and SW16. The functions of the ankyrin repeats are compatible with a role in protein-protein interactions (Bork, Proteins (1993), 17(4):363; Lambert and Bennet, Eur. J. Biochem. (1993) 211:1; Kerr et al., Current Op. Cell Biol. (1992) 4:496; Bennet et al., J. Biol. Chem (1980), 255:6424).

Basic Region Plus Leucine Zipper Transcription Factors (BZIP; Pfam Accession No. PF00170). SEQ ID NOS:108, 1714, 3931, and 4356 represent polynucleotides encoding novel members of the family of basic region plus leucine zipper transcription factors. The bZIP superfamily Hurst, Protein Prof. (1995) 2:105, and Ellenberger, Curr. Opin. Struct. Biol. (1994) 4:12) of eukaryotic DNA-binding transcription factors encompasses proteins that contain a basic region mediating sequence-specific DNA-binding followed by a leucine zipper required for dimerization. The consensus pattern for this protein family is: [KR]-x(1,3)-[RKSAQ]-N-x(2)-[SAQ](2)-x-[RKTAENQ]-x-R-x-[RK].

DEAD and DEAH box families ATP-dependent helicases (Dead box belie, Pfam Accession No. PF00270. SEQ ID NOS:38, 415, and 5756 correspond to sequence encoding a polypeptides that are members of the DEAD box family. A number of eukaryotic and prokaryotic proteins have been characterized (Schmid et al., Mol. Microbiol. (1992) 6:283; Linder et al., Nature (1989) 337:121; Wassarman et al., Nature (1991) 349:463) on the basis of their structural similarity. All are involved in ATP-dependent, nucleic-acid unwinding. All DEAD box family members of the above proteins share a number of conserved sequence motifs, some of which are specific to the DEAD family while others are shared by other ATP-binding proteins or by proteins belong to the helicases ‘superfamily’ (Hodgman, Nature (1988) 333:22 and Nature (1988) 333:578 (Errata)). One of these motifs, called the ‘D-E-A-D-box’, represents a special version of the B motif of ATP-binding proteins. Some other proteins belong to a subfamily which have His instead of the second Asp and are thus said to be ‘D-E-A-H-box’ proteins (Wassarman et al., Nature (1991) 349:463; Harosh et al., Nucleic Acids Res. (1991) 19:6331 Koonin et al., J. Gen. Virol. (1992) 73:989). The following signature patterns are used to identify member for both subfamilies: 1) [LIVMF](2)-D-E-A-D-[RKEN]-x-[LIVMFYGSTN]; and 2) [GSAH]-x-[LIVMF](3)-D-E-[ALIV]-H-[NECR].

Defensins (defensins; Pfam Accession No. PF00323. SEQ ID NO:486 corresponds to a sequence encoding a polypeptide that is a member of the mammalian defensin family. Defensins are a family of structurally related cysteine-rich peptides which are active against many Gram-positive bacteria, fungi, and enveloped viruses (Lehrer et al., ASM News (1990) 56:315-318; Lehrer et al., Cell (1991) 64:229-230; Kagan et al., Toxicology (1994) 87:131-149; Lehrer et al., Annu. Rev. Immunol. (1993) 11:105-128; White et al, Curr. Opin. Struc/Biol. (1995) 5:521-527). Some defensins inhibit corticotropoid-stimulaed corticosteroid production and also play a significant role in innate immunity to infection and neoplasia. The peptides known to belong to his family range in length from 29 to 35 amino acids and have seven invariant residues, including six cysteines that are all involved in intrachain disulfide bonds. The following consensus pattern is used to identify members of this protein family: C-x-C-x(3,5)-C-x(7)-G-x-C-x(9)-C-C.

EF Hand (Efhand; Pfam Accession No. PF00036). SEQ ID NO:4373 corresponds to a polynucleotide encoding a member of the EF-hand protein family, a calcium binding domain shared by many calcium-binding proteins belonging to the same evolutionary family (Kawasaki et al., Protein Prof. (1995) 2:305-490). The domain is a twelve residue loop flanked on both sides by a twelve residue alpha-helical domain, with a calcium ion coordinated in a pentagonal bipyramidal configuration. The six residues involved in the binding are in positions 1, 3, 5, 7, 9 and 12; these residues are denoted by X, Y, Z, —Y, —X and —Z. The invariant Glu or Asp at position 12 provides two oxygens for liganding Ca (bidentate ligand). The consensus pattern includes the complete EF-hand loop as well as the first residue which follows the loop and which seem to always be hydrophobic: D-x-[DNS]-{ILVFYW}-[DENSTG]-[DNQGHRK]-{GP}-[LIVMC]-[DENQSTAGC]-x(2)-[DE]-[LIVMFYW].

Epidermal Growth Factor (EGF; Pfam Accession No. PF00008). SEQ ID NO:3689 represents a polynucleotide encoding a member of the EGF family of proteins. The distinguishing characteristic of this family is the presence of a sequence of about thirty to forty amino acid residues found in epidermal growth factor (EGF) which has been shown to be present, in a more or less conserved form, in a large number of other proteins (Davis, New Biol. (1990) 2:410-419; Blomquist et al., Proc. Natl. Acad. Sci. U.S.A. (1984) 81:7363-7367; Barkert et al., Protein Nucl. Acid Enz. (1986) 29:54-86; Doolittle et al., Nature (1984) 307:558-560; Appella et al., FEBS Lett. (1988) 231:1-4; Campbell and Bork, Curr. Opin. Struct. Biol. (1993) 3:385-392). A common feature of the domain is that the conserved pattern is generally found in the extracellular domain of membrane-bound proteins or in proteins known to be secreted. The EGF domain includes six cysteine residues which have been shown to be involved in disulfide bonds. The main structure is a two-stranded beta-sheet followed by a loop to a C-terminal short two-stranded sheet. Subdomaims between the conserved cysteines strongly vary in length. The following consensus patterns are used to identify members of this family: C-x-C-x(5)-G-x(2)-C and C-x-C-x(s)-[GP]-[FFW]-x(4,8)-C.

Ets Domain (Ets_Cterm; Pfam Accession No. PF00178). SEQ ID NO:6 represents a polynucleotide encoding a polypeptide with C-terminal homology in the ETS domain. Proteins of this family contain a conserved domain, the “ETS-domain,” that is involved in DNA binding. The domain appears to recognize purine-rich sequences; it is about 85 to 90 amino acids in length, and is rich in aromatic and positively charged residues (Wasylyk et al., Eur. J. Biochem. (1993) 211:718). The ets gene family encodes a novel class of DNA-binding proteins, each of which binds a specific DNA sequence and comprises an ets domain that specifically interacts with sequences containing the common core tri-nucleotide sequence GGA. In addition to an ets domain native ets proteins comprise other sequences which can modulate the biological specificity of the protein. Ets genes and proteins are involved in a variety of essential biological processes including cell growth) differentiation and development, and three members are implicated in oncogenic process.

Pleckstrin Homology (PH; Pfam Accession No. PF00169). SEQ ID NOS:228 and 6001 correspond to polynucleotides encoding members of the PH family. The pleckstrin homology domain is a domain of about 100 residues that occurs in a wide range of proteins involved in intracellular signaling or as constituents of the cytoskeleton (Mayer et al., Cell (1993) 73.629-630; Haslam et al., Nature (1993) 363:309-310; Musacchio et al., Trends Biochem. Sci. (1993) 18:343-348; Gibson et al., Trends Biochem. Sci. (1994) 19:349-353 Pawson, Nature-(1995) 373:573-580; Ingley and Hemmings, J. Cell Biochem. (1994) 56:436-443, Saraste and Hyvonen, Curr. Opin. Struct. Biol. (1995) 5:463-408). All known structures of PH domains have two perpendicular anti-parallel beta sheets, followed by a C-terminal amphipathic helix. The loops connecting the beta-strands differ greatly in length, making the PH domain relatively difficult to detect (Riddihough, Nat. Struct. Biol. (1994) 1:755-757). There are no totally invariant residues within this domain.

Protein Kinase (protkinase; Pfam Accession No. PF00069). SEQ ID NOS:9, 39, 118, 229 and 4151 represent polynucleotides encoding-protein kinases, which catalyze phosphorylation of proteins in a variety of pathways, and are implicated in cancer. Eukaryotic protein kinases (Hanks et al., FASEB J. (1995) 9:576; Hunter, Meth. Enzymol. (1991) 200:3; Hanks et al., Meth. Enzymol. (1991) 200:38; Hanks, Curr. Opin. Struct. Biol. (1991) 1:369; Hanks et al., Science (1988) 241.42) belong to a very extensive family of proteins that share a conserved catalytic core common to both serine/threonine and tyrosine protein kinases. There are a number of conserved regions in the catalytic domain of protein kinases. The first region, located in the N-terminal extremity of the catalytic domain, is a glycine-rich stretch of residues in the vicinity of a lysine residue, which has been shown to be involved in ATP binding. The second region, located in the central part of the catalytic domain, contains a conserved aspartic acid residue that is important for the catalytic activity of the enzyme (Knighton et al., Science (1991) 253:407).

The protein kinase profile includes two signature patterns for this second region: one specific for serine/threonine kinases and the other for tyrosine kinases. A third profile is based on the alignment m Hanks et al. (FASEB J. (1995) 9:576) and covers the entire catalytic domain. The consensus patterns are as follows: 1) [LIV]-G-{P}-G-{P}-[FYWMCGSTNH]-[SGA]-{PW}-[LIVCAT]-{PD}-x-[GSTACLIVMFY]-x(5,18)-[LIVMFYWCSTAR]-[AIVP]-[LIVMFAGCKR]-K, where K binds ATP; 2) [LIVMFYC]-x-[HY]-x-D-[LIVMFY]-K-x(2)-N-[LIVMFYCT](3), where D is an active Site residue; and 3) [LIVMFYC]-x-[HY]-x-D-[LIVMFY]-[RSTAC]-x(2)-N-[LIVMFYC], where D is an active site residue.

Retroviral_aspartyl_protease (RVP; Pfam Accession No. PF00077). SEQ ID NO:2038 corresponds to a polynucleotide encoding a member of the Aspartyl Protease family. Aspartyl proteases also know as acid proteases, are a widely distributed family of proteolytic enzymes known to exist in vertebrates, fungi, plants, retoviruses and some plant viruses (Foltman, Essays Biochem. (1981) 17.52-84, Davies, Annu. Rev. Biophys. Chem. (1990) 19:189-215; Rao et al., Biochemistry (1991) 30:4663-4671). Most retroviral aspartyl proteases (RVP) are homodimers of a chain of about 95 to 125 amino acids. In most retroviruses, the protease is encoded as a segment of a polyprotein which is cleaved during the maturation process of the virus. RVP is generally part of the pol polyprotein and, more rarely, of the gag polyprotein. The consensus pattern is as follows: [LIVMFGAC]-[LIVMTADN]-[LIVFSA]-D-[ST]-G-[STAV]-[STAPDENQ]-x-[LIVMFSTNC]-x-[LIVMFGTA] [D is the active site residue].

Reverse Transcriptase (rvt; Pfam Accession-No. PF00078). SEQ ID NOS: 1511 and 2514 represent polynucleotides encoding reverse transcriptases, which occur in a variety of mobile elements, including retrotransposons, retoviruses, group II introns, bacterial msDNAs, hepadnaviruses, and caulimoviruses (Xiong Eickbush, EMBO J (1990) 9:3353-3362). Reverse transcriptases catalyze RNA-template-directed extension of the 3′-end of a DNA strand by one deoxnucleotide at a time and require an RNA or DNA primer.

Transforming growth factor beta like domain (TGF_beta; Pfam Accession No. PF00019). SEQ ID NO:5522 represents a polyucleotide encoding a polypeptide of the TGF-beta family, Proteins from the transforming growth factor-beta family are active as homo- or hetero-dimers with the two chains being linked by a single disulfide bond (Roberts and Sporn, In Peptide growth factors and their receptors, Handbook of Experimental Pharmacology, Vol. 95, pp 419-475, Springer Verlag, Heidelberg, (1990); Burt, Biochem. Biophys. Res. Commun. (1992) 184:590-595; Burt and Law, Prog. Growth Factor Res. (1994) 5:99-118). It is known from X-ray studies that all the other cysteines of the sequence are involved in interchain disulfide bonds (Daopin et al., Science (1992) 257:369-373). Members of this family can be recognized by the following consensus pattern: [LIVM]-x(2)-P-x-(2)-[FY]-x)(4)-C-x-G-x-C (The two C's are involved in disulfide bonds).

WD Domain (WD40), G-Beta Repeats (WD domain; Pfam Accession No. PF00400). SEQ ID NO:117 represents a member of the WD domain/G-beta repeat family. Beta-transducin (G-beta) is one of the three subunits (alpha, beta, and gamma) of the guanine nucleotide-binding proteins (G proteins) which act as intermediaries in the transduction of signals generated by transmembrane receptors (Gilman, Annu. Rev. Biochem. (1987) 56:615). The alpha subunit binds to and hydrolyzes GTP; the beta and gamma subunits are required for the replacement of GDP by GTP as well as for membrane anchoring and receptor recognition. In higher eukaryotes G-beta exists as a small multigene family of highly conserved proteins of about 340 amino acid residues. Structurally, G-beta has eight tandem repeats of about 40 residues, each containing a central Tr-Asp motif (this type of repeat is sometimes called a WD-40 repeat). The consensus pattern for the WD domain/G Beta repeat family is: [LIVMSTAC]-[LIVMFYWSTAGC] [LIMSTAG]-[LIVMSTAGC]-x(2)-[DN]-x(2)-[LIVMSTAC]-x-[LIVMFSTAG]-W-[DEN]-[LIVMFSTAGCN].

Zinc Finger, C2H2 Type (Zincfing_C₂H₂; Pfam Accession No. PF00096). SEQ ID NOS: 61, 502, 700, 847, 2034, 2054, 3403, 3524, 3653, 3723, 4688, and 4979 correspond to polynucleotides encoding members of the C₂H₂ type zinc finger protein family, which contain zinc finger domains that facilitate nucleic acid binding (Klug et al., Trends Biochem. Sci. (1987) 12:464, Evans et al., Cell (1988) 52:1; Payre et al., FEBS Lett. (1988) 234:245; Miller et al., EMBO J. (1985)-4:1609; and Berg, Proc. Natl. Acad. Sci. USA (1988) 85:99). In addition to the conserved zinc ligand residues, a number of other positions are also important for the structural integrity of the C₂H₂ zinc fingers (Rosenfeld et al., J. Biomol. Struct Dyn. (1993) 11:557). The best conserved position, which is generally an aromatic or aliphatic residue, is located four residues after the second cysteine. The consensus pattern for C₂H₂ zinc fingers is: C-x(2,4)-C-x(3)-[LIVMFYWC]-x(8)-H-x(3,5)-H. The two C's and two H's are zinc ligands.

Zinc Finger, C2HC Type (Zincfing C2HC; Pfam Accession No. PF01530). SEQ ID NO:3814 represents a member of the C2HC zinc finger family. The 18 residue C2HC domain is mainly found in the retroviral nucleocapsid protein, and is required for viral genome packaging and for the early infection process (Katz and Jentoft, Bioessays (1989) 11:176-181; Urbaneja et al., J Mol Biol. (1999) 287:59-75). In addition, the CCHC domain is found in eukaryotic proteins involved in RNA binding or single-strand DNA binding.

Zinc finger, C3HC4 type (RING finger), signature (Zincfing C3HC4; Pfam Accession No. PF00097). SEQ ID NO:3140 represents a polynucleotide encoding a polypeptide having a C3HC4 type zinc-finger signature A number of eukaryotic and viral proteins contain this signature, which is primarily a conserved cysteine-rich domain of 40 to 60 residues (Borden et al., Curr. Opin. Struct. Biol. (1996) 6'395) that binds two atoms of zinc, and is probably involved in mediating protein-protein interactions. The 3D structure of the zinc ligation system is unique to the RING domain and is referred to as the “cross-brace” motif. The spacing of the cysteines in such a domain is C-x(2)-C-x(9 to 39)-C-x(1 to 3)-H-x(2 to 3)-C-x(2)-C-x(4 to 48)-C-x(2)-C. The signature patter for the C3HC4 finger is based on the domain's central region: C-x-H-x-[LIVMFY]-C-x(2)-C-[LIVMYA].

Zinc finger, CCCH type (Zincfing CCCH; Pfam Accession No. PF00642). SEQ ID NO:877 corresponds to a polynucleotide encoding a member of the CCCH zinc finger protein family. This domain is present in many eukaryotic proteins including zinc finger proteins involved in cell cycle or growth phase-related regulation and regulatory proteins involved in regulating the response to growth factors. It has been shown that proteins containing the CCCH, zinc finger interact with the 3′ untranslated region of various mRNA (Carballo et al. Science (1998) 281:1001-1005; Lai et al. Mol. Cell Biol. (1999) 19:4311-4323) and that this domain is often present in two copies. The consensus pattern for the CCCH zinc fingers is: C-x8-C-x5-C-x3-H.

### Example 4

### Description of Libraries and Detection of Differential Expression

The relative expression levels of the polynucleotides of the invention were assessed in several libraries prepared from various sources, including cell lines and patient tissue samples. Table 1 above provides a summary of these libraries, including the shortened library name, the mRNA source used to prepared the cDNA library, the “nickname” of the library that is used in the tables below (in quotes), and the approximate number of clones in the library.

Each of the libraries is composed of a collection of cDNA clones that in turn a representative of the mRNAs expressed in the indicated mRNA source. In order to facilitate the analysis of the millions of sequences in each library, the sequences were assigned to clusters. The concept of “cluster of clones” is derived from a sorting grouping of cDNA clones based on their hybridization pattern to a panel of roughly 300 7 bp oligonucleotide probes (see Drmanac et al., Genomics (1996) 37(1):29) Random cDNA clones from a tissue library are hybridized at moderate stringency to 300 7p oligonucleotides. Each oligonucleotide has some measure of specific hybridization to that specific clone. The combination of 300 of these measures of hybridization for 300 probes equals the “hybridization signature” for a specific clone. Clones with similar sequence will have similar hybridization signatures. By developing a sorting/grouping algorithm to analyze these signatures, groups of clones in a library can be identified and brought together computationally. These groups of clones are termed “clusters” Depending on the stringency of the selection in the algorithm (similar to the stringency of hybridization in a classic library cDNA screening protocol), the “purity” of each cluster can be controlled. For example, artifacts of clustering may occur in computational clustering just as artifacts can occur in “wet-lab” screening of a cDNA library, with 400 bp cDNA fragments, at even the highest stringency. The stringency used in the implementation of cluster herein provides groups of clones that are in general from the same cDNA or closely related cDNAs. Closely related clones can be a result of different length clones of the same cDNA, closely related clones from highly related gene families, or splice variants of the same cDNA.

Differential expression for a selected cluster was assessed by first determining the number of cDNA clones corresponding to the selected cluster in the first library (Clones in 1^{st}), and the determining the number of cDNA clones corresponding to the selected cluster in the second library (Clones if 2^{nd}). Differential expression of the selected cluster in the first library relative to the second library is expressed as a “ratio” of percent expression between the two libraries. In general “ratio” is calculated by 1) calculating the percent of the selected cluster in the first library by dividing the number of clones corresponding to a selected cluster in the first library by the total number of clones analyzed from the first library; 2) calculating the percent expression of the selected cluster in the second library by dividing the number of clones corresponding to a selected cluster in a second library by the total number of clones analyzed library 3) dividing the calculated percent expression from the first library by the calculated percent expression from the second library. If the “number of clones” corresponding to a selected cluster in a library is zero, the value is set at 1 to aid in calculation. The formula used in calculating the ratio takes into account the “depth” of each of the libraries being compared; i.e., the total number of clones analyzed in each library.

In general, a polynucleotide is significantly differentially expressed between two samples when the ratio value is greater than at least about 2, preferably greater than at least about 3, more preferably greater than at least about 5, where the ratio value is calculated using the method described above. The significance of differential expression determined using a z score test (Zar, Biostatistic Analysis, Prentice, Hall. Inc. USA, “Differences Between Proportions,” pp 296-298 (1974).

Using this approach, a number Of polynucleotide sequences were identified as being differentially expressed between, for example high metastatic potential cancer tissue and low metastatic cancer cells, and between cells derived from metastatic cancer tissue and normal tissue Evaluation of the levels of expression of the genes corresponding to these sequences can be valuable in diagnosis, prognosis, and/or treatment (e.g., to facilitate rationale design of therapy, expressed sequences described herein can be therapeutic targets due to their involvement in regulation (e.g., inhibition or promotion) of development of, for example, the metastatic phenotype. For example, sequences that correspond to genes that are increased in expression in high metastatic potential cells relative to normal or non-metastatic tumor cells may encode genes or regulatory sequences involved in processes such as angiogenesis, differentiation, cell replication, and metastasis.

Detection of the relative expression levels of differentially expressed polynucleotides described herein can provide valuable information to guide the clinician in the choice of therapy. For example, a patient sample exhibiting an expression level of one or more of these polynucleotides that corresponds to a gene that is increased in expression in metastatic or high metastatic potential cells may warrant more aggressive treatment for the patient. In contrast, detection of expression levels of a polynucleotide sequence that corresponds to expression levels associated with that of low metastatic potential cells may warrant a more positive prognosis than the gross pathology would suggest.

A number of polynucleotide sequences of the present invention are differentially expressed between human microvascular endothelial cells (HMVEC) that have been treated with growth factors relative to untreated HMVEC Sequences that are differentially expressed between growth factor-treated HMVEC and untreated HMVEC can represent sequences encoding gene products involved in angiogenesis, metastasis (cell migration) and other development and oncogenic processes. For example, sequences that are more highly expressed in HMVEC treated with growth factors (such as bFGF or VEGF) relative to untreated HMVEC can serve as drug targets for chemotherapeutics, e.g., decreasing expression of such up-regulated genes or inhibiting the activity of the encoded gene product would serve to inhibit tumor cell angiogenesis. Detection of expression of these sequences in colon cancer tissue can be valuable in determining diagnostic, prognostic and/or treatment information associated with the prevention of achieving the malignant state in these tissues, and can be important in risk assessment for a patient. A patient sample displaying an increased level of one or more of these polynucleotides may thus warrant closer attention or more frequent screening procedures to catch the malignant state as early as possible.

The differential expression of the polynucleotides can thus be used as, for example, diagnostic and/or prognostic markers, for risk assessment, patient treatment and the like. These polynucleotides can also be used in combination with other molecular and/or biochemical markers.

The differential expression data for polynucleotides of the invention that have been identified as being, differentially expressed across various combinations of the libraries described above is summarized in Table 5 (inserted prior to the claims). Table 5 provides: 1) the Sequence Identification Number (“SEQ ID NO”) assigned to the polynucleotide; 2) the cluster (“CLUSTER”) to which the polynucleotide has been assigned as described above; above; 3) the library comparisons that resulted in identification of the polynucleotide as being differentially expressed (“PAIR AB”), where the cDNA libraries used are referenced by their library numbers; 4) the number of clones corresponding to the polynucleotide in the first library listed (“CLONES A”); 5) the number of clones corresponding to the polynucleotide in the second library listed (“CLONES B”); 6) the “RATIO PLUS” where the comparison resulted in a finding that the number of clones in library A is greater than the number of clones in library B; and 7) the “RATIO MINUS” where the comparison resulted in a finding that the number of clones in library B is greater than the number of clones in library A.

Detection of expression of genes that correspond to the above polynucleotides may be of particular interest in diagnosis, prognosis, risk assessment, and monitoring of treatment Furthermore, differential expression of a specific gene across multiple libraries can also be indicative of a gene whose expression is associated with, for example, suppression of the metastatic phenotype or with development of the cell toward a metastatic phenotype. For example, SEQ ID NO 3744 corresponds to a gene that is expressed at relatively higher levels in metastatized colon tumor then in normal colon tissue. Thus a relatively increased level of expression of the gene corresponding to SEQ ID NO:3744 may be used as marker of a metastatic or pre-metastatic colon cells either alone or in combination with other markers.

Some polynucleotides exhibited similar differential expression trends in libraries of different tissue origin (see, e.g., SEQ ID NO:337). These data suggest that the differential expression patterns of some genes associated with development of tumor indicate a role for those genes that is non-specific to the tissue of origin.

### Example 5

### Detection of Differential Expression Using Arrays

mRNA isolated from samples of cancerous and non colon tissue obtained from patients were analyzed to identify genes differentially expressed in cancerous and normal cells. Normal and cancerous cells collected from cryopreserved patient tissues were isolated using laser capture microdissection (LCM) techniques, which techniques are well known in the art (see, e.g., Ohyama et al. (2000) Biotechniques 29:530-6; Curran et al. (2000) Mol. Pathol. 53:64-8, Suarez-Quian et al. (1999) Biotechniques 26:328-35; Simone et al. (1998) Trends Genet 14:272-6; Conia et al. (1997) J. Clin. Lab. Anal. 11:28-38; Emmert-Buck et al. (1996) Science 274:998-1001).

Table 6 (inserted before the claims) provides information about each patient from which colon tissue samples were isolated, including: the Patient ID (“PT ID”) and Path ReportID (“Path ID”), which are numbers assigned to the patient and the pathology reports for identification purposes; the group (“Grp”) to which the patients have been assigned; the anatomical location of the tumor (“Anatom Loc”); the primary tumor size (“Size”); the primary tumor grade (“Grade”); the identification of the histopathological grade (“Histo Grade”); a description of local sites to which the tumor had invaded (“Local Invasion”); the presence of lymph node metastases (“LN Met”); the incidence of lymph node metastases (provided as a number of lymph-nodes positive for metastasis over the number of lymph nodes examined) (“Incidence Lymphnode Met”); the “Regional Lymphnode Grade”; the identification or detection of metastases to sites distant to the tumor and their location (“Dist Met & Loc”); the grade of distant metastasis (“Dist Met Grade”); and general comments about the patient or the tumor (“Comments”). Histophatology of all primary tumors indicated the tumor was adenocarcinoma except for Patient D Nos. 130 (for which no information was provided), 392 (in which greater than 50% of the cells were mucinous carcinoma), and 784 (adenosquamous carcinoma). Extranodal extensions were described in three patients, Patient ID Nos. 784, 789, and 791. Lymophovascular invasion was described in Patient ID Nos. 128, 278, 517, 534, 74, 786, 789, 791, 890, and 892. Crohn's-like infiltrates were described in seven patients, Patient ID Nos. 52, 264, 268, 392, 393, 784, and 791. Table 7 (below) provides information about the patients from whom the prostate tissue was isolated.

TABLE 7
Prostate paitent data.

| | | Normal Prostate |
|---|---|---|
| Prostate Patient ID | Tumor Gleason Grade | Description |
| 96 | 3 + 3 Adenocarcinoma | Normal prostate; |
| | | Benign hyperplasia |
| 282 | 4 + 3 Adenocarcinoma | Normal prostate; |
| | | Benign hyperplasia |
| 286 | 3 + 3 Adenocarcinoma | Normal prostate; |
| | | Benign hyperplasia |
| 294 | 3 + 4 Adenocarcinoma | Normal prostate; |
| | | Benign hyperplasia |
| 362 | 3 + 3 Adenocarcinoma | Normal prostate; |
| | | Benign hyperplasia |
| 428 | 4 + 3 Adenocarcinoma | Normal prostate; |
| | | Benign hyperplasia |
| 492 | 3 + 3 Adenocarcinoma | Normal prostate; |
| | | Benign hyperplasia |
| 492 | 3 + 3 Adenocarcinoma | Normal prostate; |
| | | Benign hyperplasia |
| 493 | 3 + 4 Adenocarcinoma | Normal prostate; |
| | | Benign hyperplasia |
| 510 | 3 + 3 Adenocarcinoma | Normal Prostate; |
| | | Benign hyperplasia |

Identification of Differentially Expressed Genes

cDNA probes were prepared from total RNA isolated from the patient cells described above. Since LCM provides for the isolation of specific cell types to provide a substantially homogenous cell sample, this provided for a similarly pure RNA sample.

Total RNA was first reverse transcribed into cDNA using a primer containing a T7 RNA polymerase promoter, followed by second strand DNA synthesis. cDNA was then transcribed in vitro to produce antisense RNA using the T7 promoter-mediated expression (see, e.g., Luo et al. (1999) Nature Med 5:117-122), and the antisense RNA was then converted into cDNA. The second set of cDNAs were again transcribed in vitro, using the T7 promoter, to provide antisense RNA. Optionally, the RNA was again converted into cDNA, allowing for up to a third round of T7-mediated amplification to produce more antisense RNA. Thus the procedure provided for two or three rounds of in vitro transcription to produce the final RNA used for fluorescent labeling.

Fluorescent probes were generated by first adding control RNA to the antisense RNA mix, and producing fluorescently labeled cDNA from the RNA starting material. Fluorescently labeled cDNAs prepared from the tumor RNA sample were compared to fluorescently labeled cDNAs prepared from normal cell RNA sample. For example, the cDNA probes from the tumor cells were labeled with Cy3 fluorescent dye (green) and the cDNA probes prepared from the tumor cells were labeled with Cy5 fluorescent dye (red), and vice versa.

each array used had an identical layout and control spot et. Each microarray was divided into two areas, each area having an array with, on each half, twelve groupings of 32×12 spots, for a total of about 9,216 spots on each array. The two areas are spotted identically which provide for at least two duplicates of each clone per array.

Polynucleotides for use on the arrays were obtained from both publicly available sources and from cDNA libraries generated from selected cell lines and patent tissues. PCR products of from about 0.5 kb to 2.0 kb amplified from these sources were spotted onto the array using a Molecular Dynamics Gen III spotter according to the manufacturer's recommendations. For polynucleotides described herein, the microarray spot contained a clone having a cDNA from which the sequence was derived. The first row of each of the 24 regions on the array had about 32 control spots, including 4 negative control spots and 8 test polynucleotides. The test polynucleotides were spiked into each sample before the labeling reaction with a range of concentrations from 2-600 pg/slide and ratios of 1:1. For each array design, two slides were hybridized with the test samples reverse-label the labeling reaction. This provided for about four duplicate measurements for each clone, two of one color, and two of the other, for each sample.

Table 8 (inserted before the claims) describes sequences present on the arrays. Table 8 includes: 1) at the SEQ ID NO of the sequence of the polynucleotide; and 2) the Spot ID, which is a unique identifier for each spot containing target sequence of interest on all arrays used.

The differential expression assay was performed by mixing equal amounts of probes from tumor cells and normal cells of the same patient. The arrays were prehybridized by incubation for about 2 hrs at 60° C. in 5×SSC/0.2% SDS/1 mM EDTA, and then washed three times in water and twice in isopropanol. Following prehybridization of the array, the probe mixture was then hybridized to the array under conditions; of high stringency (overnight at 42° C. in 50% formamide, 5×SSC, and 0.2% SDS. After hybridization, the array washed at 550C three times as follows: 1) first wash in 1× SSC/0.2% SDS; 2)-second wash in 0.1×SSC/0.2% SDS; and 3) third wash in 0.1×SSC.

The arrays were then scanned for green and red fluorescence using a Molecular Dynamics Generation III dual color laser-scanner/detector. The images were, processed using BioDiscovery Autogene software, and the data from each scan set normalized to provide for a ratio of expression relative to normal. Data from the microarray experiments was analyzed according to the algorithms described in U.S. application Ser. No. 60/252,358, filed Nov. 20, 2000, by E. J. Moler, M. A. Boyle, and F. M. Randazzo, and entitled “Precision and accuracy in cDNA microarray data,” which application is specifically incorporated herein by reference.

The experiment was repeated, this time labeling the two probes with the opposite color in order to perform the assay in both “color directions.” Each experiment was sometimes repeated with two more slides (one in each color direction). The level fluorescence for each sequence on the array expressed as a ratio of the geometric mean of 8 repliate spots/genes from the four arrays or 4 replicate spots/gene from 2 arrays or some other permutation. The data were normalized using the spiked positive controls present in each duplicated area, and the precision of this normalization was included in the final determination of the significance of each differential. The fluorescent intensity of each spot was also compared to the negative controls in each duplicated area to determine which spots have detected significant expression levels in each sample.

A statistical analysis of the fluorescent intensities was applied to each set of duplicate spots to assess the precision and significance of each differential measurement, resulting in a p-value testing the null hypothesis that there is no differential in the expression level between the tumor and normal samples of each patient. During initial analysis of the microarrays, the hypothesis was accepted if p>10⁻³ and the differential ratio was, set to 1.000 for those spots. All other spots have a significant difference in expression between the tumor and normal sample. If the tumor sample has detectable expression and the normal does not the ratio is truncated at 1000 since the value for expression in the normal sample would be zero, and the ratio would not be a mathematically useful value (e.g., infinity). If the normal sample has detectable expression and the tumor does not, the ratio is truncated to 0.001, since the value, for expression in the tumor sample would be zero and the ratio would not be a mathematically useful value. These latter two situations are referred to herein as “on/off.” Database tables were populated using 95% confidence level (p>0.05).

Table 8 (inserted before the claims) provides the results for gene products differentially expressed in the colon tumor samples relative to normal tissue samples. Table 8 includes: 1) SEQ ID NO 2) the spot identification number (“SpotID”); 3) the percentage of patients tested in which expression levels, of the gene (as detected using the corresponding clone) was at least 2-fold greater in cancerous colon tissue (primary colon tumor) than in matched normal tissue (“Colon>2×T/N,”); 4) the percentage of patients tested in which expression levels of the gene was less than or equal to one-half of the expression level in matched normal cells (“Colon<=halfx T/N”); and 5) the colon number ratios, indicating the number of patients upon which the provided ratios was based.

Table 9 below provides the data for differential expression analysis on the arrays using samples from metastazed colon tissue. In this example, the samples used for hybridization to sequences on the microarray were derived from the matched metastasized (MT) colon tissue and normal(N) colon tissues of the patients. Table 9 includes: 1) the SEQ ID NO; 2) the percentage of patients tested in which expression levels of the gene (as detected using the corresponding clone) was at least 2 fold greater in metastisized cancerous colon tissue (MT) than in matched normal tissue (“Colon>2×MT/N”); 5) the percentage of patients tested in which expression levels of the gene was less than or equal to one-half of the expression level in matched normal cells (“Colon<=halfx T/N”); and 8) the colon number ratios, indicating the number of patients upon which the provided ratios was based. The corresponding data with the same sequence of the colon tumor tissue versus matched normal colon tissue (T/N) are provide for convenience in comparison.

TABLE 9
Polynucleotides Corresponding to Differnetially Expressed
Genes in Metastasized Colon Cancer Tissue

| SEQ | | | Colon MT/N | | | |
|---|---|---|---|---|---|---|
| ID | Colon | Colon MT/N < | Num Ratios | Colon T/N > | Colon T/N < | Colon T/N |
| NO | MT/N > 2× | half× | by Clone | 2× | half× | Num Ratios |
| 217 | 40.0 | 0.0 | 5.0 | 0.0 | 50.0 | 8.0 |
| 324 | 0.0 | 40.0 | 5.0 | 0.0 | 37.5 | 8.0 |
| 1653 | 0.0 | 40.0 | 5.0 | 0.0 | 37.5 | 8.0 |
| 1972 | 40.0 | 0.0 | 5.0 | 0.0 | 42.9 | 7.0 |
| 2346 | 20.0 | 40.0 | 5.0 | 0.0 | 85.7 | 7.0 |
| 2352 | 20.0 | 80.0 | 5.0 | 0.0 | 71.4 | 7.0 |
| 2353 | 20.0 | 40.0 | 5.0 | 0.0 | 85.7 | 7.0 |
| 2647 | 0.0 | 40.0 | 5.0 | 0.0 | 62.5 | 8.0 |
| 5341 | 0.0 | 40.0 | 5.0 | 0.0 | 37.5 | 8.0 |

Table 10 below provides the data for differential expression analysis on the arrays using samples from matched cancerous and normal prostrate (PT/N). Table 10 includes 1) the SEQ ID NO; 2) the percentage of patients tested in which expression levels of the gene (as described using the corresponding clone) was at least 2-fold greater in metastisized cancerous prostate tissue (PT) than in matched normal tissue (“Con>2× PT/N”); 3) the percentage of patients tested in which expression levels of die gene was less than or equal to one-half of the expression level in match led normal cells (“Colon<=halfx PT/N”); and 4) the prostate PT/N number ratios, indicating the number of patients upon which provide ratios based. The corresponding data with the same sequences for the colon tumor versus normal (T/N) and metastasized-colon tissue versus normal (MT/N) are provided for convenience in comparison.

TABLE 10
Polynucleotides Corresponding to Differnetially Expressed Genes in Prostate Cancer Tissue

| | | | Prostate | | | | | | Colon |
|---|---|---|---|---|---|---|---|---|---|
| SEQ | Prostate | Prostate | (PT/N) | Colon | Colon | Colon T/N | Colon | Colon | MT/N |
| ID | (PT/N) > | (PT/N) < | Num | T/N > | T/N < | Num | MT/N > | MT/N < | Num |
| NO | 2× | half× | Ratios | 2× | half× | Ratios | 2× | half× | Ratios |
| 139 | 11.1 | 33.3 | 9.0 | 0.0 | 50.0 | 8.0 | | | |
| 490 | 37.5 | 12.5 | 8.0 | 0.0 | 71.4 | 7.0 | | | |
| 629 | 33.3 | 11.1 | 9.0 | | | | | | |
| 644 | 12.5 | 37.5 | 8.0 | 0.0 | 42.9 | 7.0 | | | |
| 1674 | 33.3 | 0.0 | 9.0 | | | | | | |
| 2346 | 37.5 | 25.0 | 8.0 | 0.0 | 85.7 | 7.0 | 20.0 | 40.0 | 5.0 |
| 2352 | 37.5 | 12.5 | 8.0 | 0.0 | 71.4 | 7.0 | 20.0 | 80.0 | 5.0 |
| 2420 | 22.2 | 33.3 | 9.0 | | | | | | |
| 3296 | 33.3 | 0.0 | 9.0 | 0.0 | 37.5 | 8.0 | | | |

### Example 6

### Antisense Regulation of Gene Expression

The expression of the differentially expressed genes represented by the polynucleotides in the cancerous cells can be further analyzed using antisense knockout technology to confirm the role and function of the gene product in turnorigenesis, e.g., in promoting a metastatic phenotype.

Methods for analysis using antisense technology are well known in the art. For example, a number of different oligonucleotides complementary to the mRNA generated by the differentially, expressed genes identified herein can be designed as antisense oligonucleotides and tested for their ability to suppress expression of the genes. Sets of antisense oligomers specific to each candidate target are designed using the sequences of the polynucleotides corresponding to a differentially expressed gene and the software program HYBsimulator Version 4 (available for Windows 95/Windows NT or for Power Macintosh, RNAture, Inc. 1003 Health Sciences Road, West, Irvine, Calif. 92612 USA). Factors considered when designing antisense oligonucleotides in 1) the secondary structure of oligonucleotides 2) the secondary structure of the target gene; 3) the specificity with no or minimum cross-hybridization to other expressed genes; 4) stability; 5) length and 6) terminal GC content. The antisense oligonucleotide is designed to so that it will hybridize to its target sequence under conditions of high stringency at physiological temperatures (e.g., an optimal temperature for the cells in culture to provide for hybridization in the cell, e.g., about 37° C.), but with minimal formation of homodimers.

Once synthesized and quantitated, the oligomers are screened for efficiency of a transcript knock-out in a panel of cancer cell lines. The efficiency of the knock-out is determined by analyzing mRNA levels using lightcycler quantification. The oligomers that resulted in the highest level of transcript knock-out, wherein the level was at least about 50%, preferably about 80-90%, up to 95% or more up to undetectable message, are selected for use in a cell-based proliferation assay, an anchorage independent growth assay, and an apoptosis assay.

For example, where the polynucleotide is identified as having a role in colon cancer, the ability of the corresponding designed antisense oligonucleotide to inhibit gene expression is tested through transfection into SW620 colon colorectal carcinoma cells. For each transfection mixture, a carrier molecule, preferably a lipitoid or cholesteroid, is prepared to a working concentration of 0.5 mM in water, sonicated to yield a uniform solution and filtered through a 0.45 μm PVDF membrane. The antisense or control oligonucleotide is then prepared to a working concentration of 100 μmM in sterile Milllipore water. The oligonucleotide is further diluted in OptiMEM™ (Gibco/BRL), in a microfuge tube, to, 2 μM, or approximately 20 μg oligo/ml of OPtiMEM™. In a separate microfuge tube, lipitoid or cholesteroid, typically in the amount of about 1.5-2 nmol lipitoid/μg antisense oligonucleotide, is diluted into the same volume of OPtiMEM™ used t dilute the olionucleotide. The diluted antisense oligonucleotide is immediately added to the diluted lipitoid and mixed by pipetting up and down. Oligonucleotide is added to the cells to a final concentration of 30 nM.

The level of target RNA that corresponds to a target gene of interest in the transfected cells quantitated in the cancer cell lines using the Roche LightCycler™ realtime PCR machine. Values for the target mRNA are normalized versus an internal control (e.g., beta-actin). For each 20 μl reaction, extracted RNA (generally 0.2-1 g total) is placed into a sterile 0.5 or 1.5 ml microcentrifuge tube, and water added to a total volume of 12.5 μl. To each tube 7.5 μl of a buffer/enzyme mixture is added, which is prepared by mixing (in the order listed) 2.5 μl H₂O, 2.0 μl 10× reaction buffer, 10 μl oligo dT (20 μmol), 1.0 it dNTP mix (10 mM each), 0.5 μl RNAsin® (20u) (Ambion, Inc, Hialeah, Fla.), and 0.5 μl MMLV reverse transcriptase (50u) (Ambion, Inc.). The contents are mixed by pipetting up and down, and the reaction mixture incubated at 42° C. for 1 hour. The contents of each tube are centrifuged prior to amplification.

An amplification mixture is prepared by mixing in the following order: 1×PCR buffer II, 3 mM MgCl₂, 140 μM each dNTP, 0.175 μmol each oligo, 1:50,000 dil of SYBR® Green, 0.25 mg/ml BSA, 1 unit 7Taq polymerase, and H₂O to 20 μl. (PCR buffer II is available in 10× concentration from Perkin-Elmer, Norwalk, Conn.). In 1× concentration it contains 110-M Tris pH 8.3 and 50 mM KCl. SYR® Green (Molecular Probes, Eugene, Oreg.) is a dye which fluoresces when bound to double stranded DNA. As double stranded PCR product is produced during amplification, the fluorescence from SYBR® Green increases. To each 20 μl aliquot of amplification mixture, 2 μl of template RT are added, and amplification carried out according to standard protocols.

The results can be expressed as the percent decrease in expression of the corresponding gene product relative to non-transfected cells, vehicle-only transfected (mock-transfected) cells, or cells transfected with reverse control oligonucleotides.

### Example 7

### Effect of Expression on Proliferation

The effect of gene expression on the inhibition of cell proliferation can be assessed in, for example, metastatic breast cancer cell lines (MDA-MB-231 (“231”)), SW620 colon colorectal carcinoma cells or SKOV3 cells (a human ovarian carcinoma cell line).

Cells are plated to approximately 60-80% confluency in 96-well dishes. Antisense or reverse control oligonucleotide is diluted to 2 μM in OptiMEM™ and added to OptiMEM™ into which the delivery vehicle, lipitoid 116-6 in the case of SW620 cells or 1:1 lipitoid 1:cholesteroid 1 in the case of MDA-MB-231 cells, had been diluted. The oligo/delivery vehicle mixture is then further diluted into medium with serum on the cells. The final concentration of oligonucleotide for all experiments was 300 nM, and the final ratio of oligo to delivery vehicle for all experiments is 1.5 nmol lipitoid/μg oligonucleotide.

Antisense oligonucleotides are prepared as described above (see Example 6). Cells are transfected overnight at 37° C. and the transfection mixture replaced with fresh medium the next morning. Transfection is carried out as described above in Example 3.

Those antisense oligonucleotides that inhibit proliferation represent genes that lay a role in production or maintenance of the cancerous phenotype.

### Example 8

### Effect of Gene Expression on Colony Formation

The effect of gene expression upon colony formation of, for example SVS20 cells, SKOV3 cells, and MD-MBA-231 cells can be tested in a soft agar assay. Soft agar assays are conducted by first-establishing a bottom layer of 2 ml of 0.6% agar in media plated fresh within a few hours of layering on the cells. The cell layer is formed on the bottom layer by removing cells transfected as described above from plates using 0.05% trypsin and washing twice in media. The cells are counted in a Coulter counter, and resuspended to 10⁶ per ml in media. 10 μl aliquots are placed with media in 96-well plates (to check counting with WST1), or diluted further for the soft agar assay. 2000 cells are plated in 800 μl 0.4% agar in duplicate wells above 0.6% agar bottom layer. After the cell layer agar solidifies, 2 ml of media is dribbled on top and antisense or reverse control oligo (produced as described in Example 6) added without delivery vehicles. Fresh media and oligos are added every 3-4 days: Colonies usually are expected to form in 10 days to 3 weeks. Fields of colonies are counted by eye. Wst-1 metabolism values can be used to compensate for small differences in starting cell number. Larger fields can be scanned for visual record of differences.

Those antisense oligonucleotides that inhibited colony formation represent genes that play a role in production or maintenance of the cancerous phenotype.

### Example 9

### Induction of Cell Death upon Depletion of Polypeptides by Depletion of mRNA (“Antisense Knockout”)

In order to assess the effect of depletion of a target message upon cell death SW620 cells or other cells derived from a cancer of interest are transfected for proliferation assays. For cytotoxic effect in the presence of cisplatin (cis), the same protocol is followed but cells are left in the presence of 2 μM drug. Each day, cytotoxicity was monitored by measuring the amount of LDH enzyme released in the medium due to membrane damage. The activity of LDH is measured using the Cytotoxicity Detection Kit from Roche Molecular Biochemicals. The data is provided as a ratio of LDH released in the medium vs. the total LDH present in the well at the same time point and treatment (rLDH/tLDH). DA positive control using antisense and reverse control oligonucleotides for BCL2 (a known anti-apoptotic gene) is included; loss of message for BCL2 leads to an increase in cell death compared with treatment with the control oligonucleotide (background cytotoxicity due to transfection).

### Example 10

### Functional Analysis of Gene Products Differentially Expressed in Cancer

The gene products of sequences of a gene differentially expressed in cancerous cells can be further analyzed to confirm the role and function of the gene product in tumorigenesis, e.g., in promoting or inhibiting development of a metastatic phenotype. For example, the function of gene products corresponding to genes identified herein can be assessed by blocking function of the gene products in the cell. For example, where the gene product is secreted or associated with a cell surface membrane, blocking antibodies can be generated and added to cells to examine the effect upon the cell phenotype in the context of, for example, the transformation of the cell to a cancerous, particularly a metastatic, phenotype.

Where the gene product of the differentially expressed genes identified herein exhibits sequence homology to a protein of known function (e.g., to a specific kinase or protease) and/or to a protein family of known function (e.g., contains a domain or other consensus sequence present in a protease family or in a kinase family), then the role of the gene product in tumorigenesis, as well as the activity of the gene product, can be examined using small molecules that inhibit or enhance function of the corresponding protein or protein family.

Additional functional assays include, but are not necessarily limited to, those that analyze the effect of expression of the corresponding gene upon cell cycle and cell migration. Methods for performing such assays are well known in the art.

### Example 11

### Contig Assembly and Additional Gene Characterization

The sequences of the polynucleotides provided in the present invention can be used to extend the sequence information of the gene to which the polynucleotides correspond (e.g., a gene, or mRNA encoded by the gene having a sequence the polynucleotide descried herein). This expanded sequence information can in turn be used to further characterize the corresponding gene, which in turn provides additional information about the nature of the gene product (e.g., the normal function of the gene product). The additional information can serve to provide additional evidence of the gene product's use as a therapeutic target, and provide further guidance as to the types of agents that can modulate its activity.

For example, a contig can be assembled using the sequence of a polynucleotide described herein. A “contig” is a contiguous sequence of nucleotides that is assembled from nucleic acid sequences having overlapping (e.g., shared or substantially similar) sequence information. The sequences of publicly-available ESTs (Expressed Sequence Tags) and the sequences of various clones from several cDNA libraries synthesized at Chiron were used in the contig assembly. The contig is assembled using the soft are program Sequencher, version 4.05, according to the manufacturer's instructions. The resulting contig can then be used to search both the public databases as well as databases internal to the applications to match the polyucleotide contiged with homology data and/or differential gene expressed data.

The sequence information obtained in the contig assembly described above can be used to obtain a consensus sequence derived from the contig using the Sequenchler program. The consensus sequence can then be used as a query sequence in a BLASTN search of the DGTI DoubleTwist Gene Index (DoubleTwist, Oakland, Calif.), which contains all the EST and non-redundant sequence in public databases. Alternatively, a sequence of a polynucleotide described herein can be used directly as a query sequence in a BLASTN search of the DGTI DoubleTwist Gene Index.

Through contig assembly and the use of homology searching software programs, the sequence information provided herein can be readily extended to confirm or confirm a predicted, gene having the sequence of the polynucleotides described in the present invention. Further the information obtained can be used to identify the function of the gene product of the gene corresponding to the polynucleotides described herein. Wile not necessary to the practice of the invention, identification of the function of the corresponding gene, can provide guidance in the design of therapeutics that target the gene to modulate its activity and modulate the cancerous phenotype (e.g., inhibit metastasis, proliferation, and the like).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Those skilled in the art will recognize, or be able to ascertain using not more than routine experimentation, many equivalents to the specific embodiments of the invention described herein. Such specific embodiments and equivalents are intended to be encompassed by the following claims.

Deposit Information

A deposit of the biological materials in the tables referenced below was made with the American Type Culture Collection 10801 University Blvd., Manasas, Va. 20110-2209, under the provisions of the Budapest Treaty on or before the filing date of the present application. The accession number indicated is assigned after successful viability testing, and the requisite fees were paid. Access to said cultures will be available during pendency of the patent application to one determined by the Commissioner to be entitled such under 37 C.F.R. §1.14 and 35 U.S.C. §122. All restriction on availability of said cultures to the public will be irrevocably removed upon the granting of a patent based upon the application. Moreover, the designated deposits will be maintained for a period of thirty (30) years from the date of deposit, or for five (5) years after the last request for the depositor for the enforceable life of the U.S. patent, whichever is longer. Should a culture become nonviable or be inadvertently destroyed or, in the case of plasmid-containing stains, lose its plasmid, it will be replaced with a viable culture(s) of the same taxonomic description.

These deposits are provided merely as a convenience to those of skill in the art, and are not an admission that a deposit is required. A license may be required to make, use, or sell the deposited materials, and no such license is hereby granted. The deposit below was received by the ATCC on or before the filing date of the present application.

TABLE 11
Cell Lines Deposited with ATCC

| | | ATCC | CMCC |
|---|---|---|---|
| Cell Line | Deposit Date | Accession No. | Accession No. |
| KM12L4-A | Mar. 19, 1998 | CRL-12496 | 11606 |
| Km12C | May 15, 1998 | CRL-12533 | 11611 |
| MDA-MB- | May 15, 1998 | CRL-12532 | 10583 |
| 231 | | | |
| MCF-7 | Oct. 9, 1998 | CRL-12584 | 10377 |

In addition, pools of selected clones, as well as libraries containing specific clones, were assigned an “ES” number (internal reference) and deposited with the ATCC. Table 13 below provides the ATCC Accession Nos of the ES deposits all of which were deposited on or before Jun. 13, 2000. The names of the clones contained within each of these deposits are provided in the tables numbered 22 and greater (inserted before the claims).

TABLE 12
Pools of Clones and Libraries Deposited
with ATCC on or before Jun. 13, 2000.

| Library No. | CMCC No. | ATCC Accession No. |
|---|---|---|
| ES168 | 5276 | PTA-2027 |
| ES169 | 5277 | PTA-2028 |
| ES170 | 5284 | PTA-2029 |
| ES171 | 5285 | PTA-2030 |
| ES172 | 5286 | PTA-2031 |
| ES173 | 5287 | PTA-2032 |
| ES174 | 5288 | PTA-2033 |
| ES175 | 5289 | PTA-2034 |
| ES176 | 5290 | PTA-2035 |
| ES177 | 5291 | PTA-2036 |
| ES178 | 5292 | PTA-2037 |
| ES179 | 5293 | PTA-2038 |
| ES180 | 5294 | PTA-2039 |
| ES181 | 5295 | PTA-2040 |
| ES182 | 5296 | PTA-2041 |
| ES183 | 5297 | PTA-2042 |
| ES184 | 5298 | PTA-2043 |
| ES185 | 5299 | PTA-2044 |
| ES186 | 5301 | PTA-2045 |
| ES187 | 5302 | PTA-2046 |
| ES188 | 5303 | PTA-2047 |
| ES189 | 5304 | PTA-2052 |
| ES190 | 5305 | PTA-2053 |
| ES191 | 5306 | PTA-2054 |
| ES192 | 5307 | PTA-2055 |
| ES193 | 5308 | PTA-2056 |
| ES194 | 5309 | PTA-2057 |
| ES195 | 5310 | PTA-2058 |
| ES196 | 5311 | PTA-2059 |
| ES197 | 5312 | PTA-2060 |
| ES198 | 5313 | PTA-2061 |
| ES199 | 5314 | PTA-2062 |
| ES200 | 5315 | PTA-2048 |
| ES201 | 5316 | PTA-2049 |
| ES202 | 5317 | PTA-2063 |
| ES203 | 5318 | PTA-2064 |
| ES204 | 5319 | PTA-2065 |
| ES205 | 5320 | PTA-2066 |
| ES206 | 5321 | PTA-2067 |
| ES207 | 5322 | PTA-2068 |
| ES208 | 5253 | PTA-2050 |
| ES209 | 5324 | PTA-2051 |

Table 13 (inserted before the claims) provides the names of the clones in each of the above libraries.

Retrieval of Individual Clones from Deposit of Pooled Clones. Where the ATCC deposit is composed of a pool of cDNA clones or a library of cDNA clones, the deposit was prepared by first transfecting each of the clones into separate bacterial cells. The clones in the pool or library were then deposited as a pool of equal mixtures in the composite deposit. Particular clones can be obtained from the composite deposit using methods well known in the art. For example, a bacterial cell containing a particular clone can be identified by isolating single colonies, and identifying colonies containing the specific clone though standard colony hybridization techniques, using an oligonucleotide probe or probes be designed to specifically hybridize to a sequence of the clone, insert (e.g., a probe based upon unmasked sequence of the encoded polynucleotide having the indicated SEQ ID NO). The probe should be designed to have a Tₘ of approximately 80° C. (assuming 2° C. for each A or T and 4° C. for each G or C). Positive colonies can the be picked, grown in culture, and the recombinant clone isolated. Alternatively, probes designed in this manner can be used to PCR to isolate a nucleic acid cDNA from the deposited culture pool, and using the probes in PCR reactions to produce an amplified product haying the corresponding desired polynucleotide sequence.

LENGTHY TABLE REFERENCED HERE
US20070243176A1-20071018-T00001
Please refer to the end of the specification for access instructions.

LENGTHY TABLE REFERENCED HERE
US20070243176A1-20071018-T00002
Please refer to the end of the specification for access instructions.

LENGTHY TABLE REFERENCED HERE
US20070243176A1-20071018-T00003
Please refer to the end of the specification for access instructions.

LENGTHY TABLE REFERENCED HERE
US20070243176A1-20071018-T00004
Please refer to the end of the specification for access instructions.

LENGTHY TABLE REFERENCED HERE
US20070243176A1-20071018-T00005
Please refer to the end of the specification for access instructions.

LENGTHY TABLE REFERENCED HERE
US20070243176A1-20071018-T00006
Please refer to the end of the specification for access instructions.

LENGTHY TABLE REFERENCED HERE
US20070243176A1-20071018-T00007
Please refer to the end of the specification for access instructions.

LENGTHY TABLE REFERENCED HERE
US20070243176A1-20071018-T00008
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070243176A1). An electronic copy of the table will also be available from the USPTO upon request and payment of the fee set forth in 37 CFR 1.19(b)(3).

We claim:

1. An isolated polynucleotide comprising a nucleotide sequence which hybridizes under stringent conditions to a sequence selected from the group consisting of SEQ ID NOS: 1-6010.

2. An isolated polynucleotide comprising at least 15 contiguous nucleotides of a nucleotide sequence having at least 90% sequence identity to a sequence selected from the group consisting of:
SEQ ID NOS: 1-6010, a degenerate variant of SEQ ID NOS: 1-6010, an antisense of SEQ ID NOS: 1-6010, and a complement of SEQ ID NOS: 1-6010.

3. An isolated polynucleotide comprising at least 15 contiguous nucleotides of a nucleotide sequence selected from the group consisting of: SEQ ID NOS:1-6010, a degenerate variant of SEQ ID NOS:1-6010, an antisense of SEQ ID NOS:1-6010, and a complement of SEQ ID NOS: 1-6010.

4. The isolated polynucleotide of claim 3, wherein the polynucleotide comprises at least 100 contiguous nucleotides of the nucleotide sequence.

5. The isolated polynucleotide of claim 3, wherein the polynucleotide comprises at least 200 contiguous nucleotides of the selected nucleotide sequence.

6. An isolated polynucleotide comprising a nucleotide sequence of at least 90% sequence identity to a sequence selected from the group consisting of: SEQ ID NOS: 1-6010, a degenerate variant of SEQ ID NOS:1-6010, an antisense of SEQ ID NOS:1-6010, and a complement of SEQ ID NOS:1-6010.

7. The isolated polynucleotide of claim 6, wherein the polynucleotide comprises a nucleotide sequence of at least 95% sequence identity to the selected nucleotide sequence.

8. The isolated polynucleotide of claim 6, wherein the polynucleotide comprises a nucleotide sequence that is identical to the selected nucleotide sequence.

9. A polynucleotide comprising a nucleotide sequence of an insert contained in a clone deposited as ATCC Accession No. PTA-2027, PTA-2028, PTA-2029, PTA-2030, PTA-2031, PTA-2032, PTA-2033, PTA-2034, PTA-2035, PTA-2036, PTA-2037, PTA-2038, PTA-2039, PTA-2040, PTA-2041, PTA-2042, PTA-2043, PTA-2044, PTA-2045, PTA-2046, PTA-2047, PTA-2050, PTA-2051, PTA-2052, PTA-2053, PTA-2054, PTA-2055, PTA-2056, PTA-2057, PTA-2058, PTA-2059, PTA-2060, PTA-2061, PTA-2062, PTA-2048, PTA-2049, PTA-2063, PTA-2064, PTA-2065, PTA-2066, PTA-2067, or PTA-2068.

10. An isolated cDNA obtained by the process of amplification using a polynucleotide comprising at least 15 contiguous nucleotides of a nucleotide sequence of a sequence selected from the group consisting of SEQ ID NOS: 1-6010.

11. The isolated cDNA of claim 10, wherein the polynucleotide comprises at least 25 contiguous nucleotides of the selected nucleotide sequence.

12. The isolated cDNA of claim 10, wherein the polynucleotide comprises at least 100 contiguous nucleotides of the selected nucleotide sequence.

13. The isolated cDNA of claims 10, 11, or 12, wherein amplification is by polymerase chain reaction (PCR) amplification.

14. An isolated recombinant host cell containing the polynucleotide according to claims 1, 2, 3, 6, 9, or 10.

15. An isolated vector comprising the polynucleotide according to claims 1, 2, 3, 6, 9, or 10.

16. A method for producing a polypeptide, the method comprising the steps of:
culturing a recombinant host cell containing the polynucleotide according to claims 1, 2, 3, 6, 9, or 10., said culturing being under conditions suitable for the expression of an encoded polypeptide;
recovering the polypeptide from the host cell culture.

17. An isolated polypeptide encoded by the polynucleotide according to claims 1, 2, 3, 6, 9, or 10.

18. An antibody that specifically binds the polypeptide of claim 17.

19. A method of detecting differentially expressed genes correlated with a cancerous state of a mammalian cell, the method comprising the step of:
detecting at least one differentially expressed gene product in a test sample derived from a cell suspected of being cancerous, where the gene product is encoded by a gene comprising an identifying sequence of at least one of SEQ ID NOS: 1-6010;
wherein detection of the differentially expressed gene product is correlated with a cancerous state of the cell from which the test sample was derived.

20. A library of polynucleotides, wherein at least one of the polynucleotides comprises the sequence information of the polynucleotide according to claims 1, 2, 3, 6, 9, or 10.

21. The library of claim 20, wherein the library is provided on a nucleic acid array.

22. The library of claim 20, wherein the library is provided in a computer-readable format.

23. A method of inhibiting tumor growth by modulating expression of a gene product, the gene product being encoded by a gene identified by a sequence selected from the group consisting of SEQ ID NOS:1-6010.
